(19) United States
(12) Patent Application Publication
(10) Pub. No.: US 2020/0390496 A1
(43) Pub. Date: Dec. 17, 2020

(54) ELECTROMAGNETIC RADIATION ABLATION TIPS MADE OF MAGNETIC MATERIALS

(71) Applicant: AVOLT, LLC, Ogden, UT (US)

(72) Inventors: Timothy Houden, Ogden, UT (US); Brett Peterson, West Bountiful, UT (US); Kent F. Beck, Layton, UT (US)

(73) Assignee: AVOLT, LLC, Ogden, UT (US)

(21) Appl. No.: 16/900,935

(22) Filed: Jun. 14, 2020

Related U.S. Application Data
(60) Provisional application No. 62/861,748, filed on Jun. 14, 2019.
(60) Provisional application No. 62/883,584, filed on Aug. 6, 2019.
(60) Provisional application No. 62/885,057, filed on Aug. 9, 2019.

(51) Int. Cl. A61B 18/14

(52) U.S. Cl. CPC A61B 18/1492; A61B 2018/00577

(57) ABSTRACT

Embodiments described herein relate to methods, systems, and devices that are used in the radio frequency ablation of tissues in a medical treatment. Probe tips may include an expandable balloon or basket with an active RF treatment area localized on a particular portion of the expandable balloon or basket. The broad or localized active treatment region on the balloon or basket allows a medical practitioner to more completely provide treatment to a region of target tissue, a volume within a tissue such as a bone, or a surface of a tissue such as a bone. Probe tips may also use magnetic materials which reduce the risk of injuring surrounding tissues adjacent to tissues under treatment.

### PRIORITY

The present application claims the benefit of U.S. Provisional Application Ser. No. 62/861,748, filed Jun. 14, 2019, which is herein incorporated by reference in its entirety. The present application claims the benefit of U.S. Provisional Application Ser. No. 62/883,584, filed Aug. 6, 2019, which is herein incorporated by reference in its entirety. The present application claims the benefit of U.S. Provisional Application Ser. No. 62/885,057, filed Aug. 9, 2019, which is herein incorporated by reference in its entirety.

### THE FIELD OF THE INVENTION

The described embodiments relate generally to radio frequency surgical ablation probes. Described embodiments also relate to deployable ablation probes and deployable ablation probes that have tips which may be used to ablate a targeted tissue area without ablating an adjacent tissue area.

### BACKGROUND

Many patients present with tissue that needs to be treated. For example, physicians may need to treat vascular tissues, central nervous system tissues, peripheral nervous system tissues, peripheral nerves, tumors, malignant tissues, and nonmalignant tissues, as well as other tissues in the body. As medical imaging and diagnostic methods improve, it is possible to treat conditions at earlier stages. An advantage of earlier detection is that the tissues that require treatment may be smaller. These tissues may be located in more remote locations within the body than previously detectable. As the tissues that require treatment get smaller and/or more remotely located within the body, there is a need for tools which may be used to treat tissues which are small, precise, and remotely located.

### SUMMARY OF THE INVENTION

Embodiments of the invention include methods, systems, and devices for treating tissue with oscillating electromagnetic radiation, in particular radio frequency electromagnetic radiation. The radio frequency electromagnetic radiation may also be identified as radio frequency (RF) energy. More specifically, this application is directed to methods, systems, and devices for use in radio frequency ablation (RFA) for the treatment of various medical conditions, and, in particular, the use of magnetic (MM) materials in the tips of RFA probes (e.g., stylets). The MM materials in the tips may comprise single elements, alloys, or layered materials.

One embodiment described herein takes the form of an electromagnetic radiation tissue treatment system comprising: an electromagnetic radiation probe, comprising: an electrode tip comprising a magnetic material; and an electrode conducting portion structurally coupled to and in electrical communication with the electrode tip, the electrode tip being at a distal end of the electrode conducting portion; and an oscillating electromagnetic energy source electrically connected to a proximal end of the electrode conducting portion.

The electromagnetic radiation tissue treatment system may include a cannula, and the cannula may be bent, straight, curved, wide, narrow, sharp, or blunt. The electromagnetic radiation probe may be moveably positioned within the cannula such that the electrode tip is proximal to a distal end of the cannula. The electrode conducting portion may have electrical leads that proceed from the proximal end of the electrode conducting portion and are in electrical communication with the oscillating electromagnetic energy source. The oscillating electromagnetic energy source provides an oscillating electrical signal that generates an electromagnetic field around the electrode tip. The electromagnetic field generated around the electrode tip may create a lesion in the tissue. The oscillating electrical signal may be a radio frequency signal, and the oscillating electrical signal may have a frequency of between about 30 hertz and about 300 gigahertz. Furthermore, the oscillating electrical signal may have a frequency of between about 350 kilohertz and about 500 kilohertz. In embodiments, the MM tips may have more than one tine.

In embodiments of the electromagnetic radiation tissue treatment system, the electrode tip may be introduced into nervous system tissue and other tissue and the oscillating electrical signal may have a frequency of between about 350 kilohertz and about 500 kilohertz. The electromagnetic field generated around the electrode tip creates a lesion in the nervous tissue.

Another embodiment described herein takes the form of a radio frequency ablation probe, comprising: a hollow probe body; and an electrode positioned within the hollow probe body that can travel freely along a length of the hollow probe body, the electrode comprising: an electrode conducting portion in electrical communication with a radio frequency power supply at a proximal end of the electrode conducting portion; and a magnetic portion in electrical communication with the electrode conducting portion and at a distal end of the electrode conducting portion forming a magnetic electrode tip. The magnetic portion may be formed from a ferromagnetic material comprising at least one or more of iron, nickel, cobalt, neodymium, dysprosium, or gadolinium.

In another embodiment, the magnetic portion may be formed from a magnetic ceramic material. One example of a magnetic ceramic material is ferrite material comprising large proportions of iron (III) oxide (Fe₂O₃) blended with small proportions of one or more additional metallic elements, such as barium, manganese, nickel, or zinc. The choice of the elements comprising the ferrite material in particular, or another magnetic ceramic material, in general, is not limited to iron, barium, manganese, nickel, and zinc, but may include other elements, such as cobalt, manganese, and strontium as needed to tailor the desired magnetic and conducting properties of the magnetic electrode tips. In embodiments, the magnetic portion may be comprised of a mixture of ferromagnetic and ferrite materials.

In an embodiment of the radio frequency ablation probe, the magnetic portion comprises: a magnetic material core in electrical communication with the electrode conducting portion; and a non-magnetic material encasing the magnetic material core and coupled to a peripheral portion of the distal end of the electrode conducting portion.

In an embodiment of the radio frequency ablation probe, the radio frequency power supply may be a first radio frequency power supply, and the magnetic portion comprises: a magnetic material core in electrical communication with the electrode conducting portion; a coil of a first non-magnetic material wrapped around the magnetic material core and in electrical communication with a second radio frequency power supply, where the coil of the first non-magnetic material is electrically isolated from the magnetic portion; and a second non-magnetic material encasing the magnetic material core and the first non-magnetic material, where the second non-magnetic material is electrically isolated from the magnetic material core and the first non-magnetic material, and the second non-magnetic material is coupled to a peripheral portion of the distal end of the electrode conducting portion.

The first non-magnetic material may comprise a first metal or metallic alloy including at least one or more of aluminum, copper, lead, nickel, tin, titanium, zinc, niobium, tantalum, vanadium, gold, silver, or palladium. The second non-magnetic material may comprise a second metal or metallic alloy including at least one or more of aluminum, copper, lead, nickel, tin, titanium, zinc, niobium, tantalum, vanadium, gold, silver, or palladium.

In embodiments, the hollow probe body is rigid and has a sharp point at a distal end of the hollow probe body that can puncture many types of tissue, including bone; and the electrode may be moved along the hollow probe body so the magnetic electrode tip emerges from the sharp point of the hollow probe body and is used for radiofrequency ablation. The hollow probe body may define a straight tip or a curved tip.

Still another embodiment described herein takes the form of a method of treating tissue with electromagnetic radiation, comprising: selecting an electromagnetic radiation probe comprising a magnetic material electrode tip; providing oscillating electrical signals to the magnetic material electrode tip; and contacting the tissue with the electromagnetic radiation delivered by the magnetic material electrode tip to treat the tissue.

In embodiments, the electromagnetic radiation probe may also include a cannula and an electrode movably positioned within the cannula. The electrode may also include an electrode conducting portion in electrical communication with a power supply at a proximal end of the electrode conducting portion. The magnetic material electrode tip may be in electrical communication with the electrode conducting portion. The magnetic material electrode tip may be rigidly attached to the electrode conducting portion. The electrode tip may define a sharp point at a distal end of the electrode.

In embodiments, the electromagnetic radiation probe may further include an expandable balloon that can deliver the electromagnetic radiation to a larger region of tissue than may be accessed by a probe without the balloon. The balloon is flexible and can adapt to the shape required by the type of tissue being treated. The balloon may expand to engage with a larger region of tissue. The balloon also may expand to around a cylindrical tissue, such as a nerve. The balloon may expand within a volume of tissue, such as bone marrow, muscle, skin, or a bladder. In some treatment situations, the balloon may adapt to one or more of the treatment shapes described above. In embodiments, contacting the tissue comprises introducing the magnetic material electrode tip adjacent to the tissue, or into the tissue, and providing the electromagnetic radiation to the tissue to treat the tissue. Sample types of tissue treatments include lesioning, cutting, cauterization, ablation, necrosing, and coagulation. In embodiments, tissue treatments may include bringing the electromagnetic radiation probe adjacent to the tissue, but not physically contacting the tissue under treatment. The types of tissue treated include connective tissues, cartilage, muscle, cardiac tissue, adipose, vascular tissues, central nervous system tissues, peripheral nervous system tissues, peripheral nerves, tumors, malignant tissues, and nonmalignant tissues.

### BRIEF DESCRIPTION OF THE DRAWINGS

The disclosure will be readily understood by the following detailed description in conjunction with the accompanying drawings, wherein like reference numerals designate like elements.

FIG. 1A illustrates a user interacting with a sample RFA system showing certain components of a sample embodiment.

FIG. 1B is an RFA probe diagram illustrating certain components of a sample embodiment.

FIG. 2 is a block diagram of an embodiment of a sample RFA system.

FIG. 3A illustrates an embodiment of an RFA probe with an RFA tip in a retracted position.

FIG. 3B illustrates an embodiment of an RFA probe with an RFA tip in an extended position.

FIG. 3C illustrates an embodiment of an RFA probe with an RFA tip in an extended position.

FIG. 3D illustrates an embodiment of an RFA probe with an RFA tip in an extended position.

FIG. 4A illustrates an embodiment of an RFA tip.

FIG. 4B illustrates an embodiment of an RFA tip.

FIG. 4C illustrates an embodiment of an RFA tip.

FIG. 4D illustrates an embodiment of an RFA tip.

FIG. 5A illustrates an embodiment of an RFA probe with an RFA tip having two tines in a retracted position.

FIG. 5B illustrates an embodiment of an RFA probe with an RFA tip having two tines in an extended position.

FIG. 5C illustrates an embodiment of an RFA probe with an RFA tip having two tines in an extended position.

FIG. 5D illustrates an embodiment of an RFA probe with an RFA tip having two tines in an extended position.

FIG. 5E illustrates an embodiment of an RFA probe with an RFA tip having two tines in an extended position showing a potential overlap of RF field lines.

FIG. 6A illustrates an embodiment of an RFA probe with an RFA tip and an expandable balloon.

FIG. 6B illustrates an embodiment of an RFA probe with an RFA tip and an expandable balloon.

FIG. 6C illustrates an embodiment of an RFA probe with an RFA tip and an expandable balloon.

FIG. 7A illustrates an embodiment of an RFA probe with an RFA tip and an expandable balloon prior to the balloon physically engaging with a tissue under treatment.

FIG. 7B illustrates an embodiment of an RFA probe with an RFA tip and an expandable balloon after the balloon is physically engaging with a tissue under treatment.

FIG. 8A is a drawing of an example RFA system.

FIG. 8B is a drawing of an example RFA probe.

FIG. 9 is a drawing of a probe tip and cannula with the probe tip in a retracted position.

FIG. 10 is a drawing of a probe tip and cannula with the probe tip in an extended or deployed position.

FIG. 11 is a drawing of a probe tip and cannula with the probe tip in an expanded position.

FIG. 12 shows a side view drawing of an expandable probe tip.

FIG. 13 shows a distal end view drawing of the expandable probe tip of FIG. 12.

FIG. 14 shows a side view drawing of the expandable probe tip of FIG. 12 in an expanded state.

FIG. 15 shows a distal end view drawing of the expandable probe tip of FIG. 12 in an expanded state.

FIG. 16 shows a side view drawing of an expandable probe tip similar to that of FIG. 12 with an alternate balloon and electrode configuration in an expanded state.

FIG. 17 shows a side view drawing of an expandable probe tip.

FIG. 18 shows a distal end view drawing of the expandable probe tip of FIG. 17.

FIG. 19 shows a side view drawing of the expandable probe tip of FIG. 17 in an expanded state.

FIG. 20 shows a distal end view drawing of the expandable probe tip of FIG. 17 in an expanded state.

FIG. 21 shows a side view drawing of an expandable probe tip similar to that of FIG. 17 with an alternate balloon and electrode configuration in an expanded state.

FIG. 22 shows a cross sectional drawing of an electrode tip as shown in FIG. 16 or FIG. 21.

FIG. 23 shows a cross sectional drawing of an electrode tip with an alternate balloon configuration.

FIG. 24 shows a cross sectional drawing of the electrode tip of FIG. 23 in an expanded state.

FIG. 25 shows a side view drawing of an expandable probe tip in an expanded state.

FIG. 26 shows a distal end view drawing of the expandable probe tip of FIG. 25 in an expanded state.

FIG. 27A shows a side view drawing of an expandable probe tip.

FIG. 27B shows a side view drawing of an expandable probe tip.

FIG. 28A shows a side view drawing of the expandable probe tip of FIG. 27 in an expanded state.

FIG. 28B shows a side view drawing of the expandable probe tip of FIG. 27 in an expanded state.

FIG. 29 shows a distal end view drawing of the expandable probe tip of FIG. 27 in an expanded state.

FIG. 30 shows a side view drawing of an expandable probe tip.

FIG. 31 shows a drawing of an example surgical use of the probe.

FIG. 32 shows a drawing of an example surgical use of the probe.

FIG. 33 shows a drawing of an example surgical use of the probe.

FIG. 34 shows a drawing of an example surgical use of the probe.

FIG. 35 shows a drawing of an example surgical use of the probe.

FIG. 36 shows a drawing of an example surgical use of the probe.

The use of cross-hatching or shading in the accompanying figures is generally provided to clarify the boundaries between adjacent or abutting elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Unless otherwise noted, the drawings have been drawn to scale. The proportions and dimensions (either relative or absolute) of the various features and elements (and collections and groupings thereof) and the boundaries, separations, and positional relationships presented there between, are provided in the accompanying figures to facilitate an understanding of the various embodiments described herein. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be used in connection with other embodiments whether or not explicitly described. The particular features, structures or characteristics may be combined in any suitable combination and/or sub-combinations in one or more embodiments or examples.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be such as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

In some instances, a structure (e.g. a probe or a probe tip) has been identified in different figures using different reference numerals. It is understood that, unless otherwise noted, the description of an element in one figure applies to that element as shown in other figures and that features shown in combination with an element may also be used in combination with the element as described in other figures. Unless incompatible or otherwise noted, the various structures described herein may be used in any combination with other structures described herein.

### DETAILED DESCRIPTION

Reference will now be made in detail to representative embodiments illustrated in the accompanying drawings. It should be understood that the following descriptions are not intended to limit the embodiments to one preferred implementation. To the contrary, the described embodiments are intended to cover alternatives, modifications, and equivalents as can be included within the spirit and scope of the disclosure and as defined by the appended claims.

Embodiments described herein generally reference a radio frequency ablation (RFA) system and, in particular, an RFA probe. An RFA probe may include at least a hollow probe body and an electrode positioned within the hollow probe body. The electrode may include a radio frequency (RF) tip at the distal end of the electrode and electrical connector cables at the proximal end, where the electrical connector cables are plugged into an oscillating energy source. In a given treatment setting, a medical service provider may manipulate the RFA probe to insert the RF tip into, or position the RF tip adjacent to, a tissue under treatment. The oscillating energy is conducted from the oscillating energy source through the connecting cables, through the electrode, and to the RF tip, where the RF tip delivers energy, in the form of oscillating electromagnetic radiation, generated by the oscillating energy source to the tissue under treatment.

As used in this document, the term “tips” refers to the tips of RFA probes. Depending on the material comprising the “tips,” the tips used in RFA may be identified in the various embodiments as radio frequency (RF) tips, RFA tips, electrode tips, magnetic electrode tips, magnetic material (MM) electrode tips, or MM tips. More specifically, the terms RF tips, RFA tips, and electrode tips refer broadly to tips which may comprise any kind of material, including magnetic and non-magnetic materials. The terms magnetic electrode tips, MM electrode tips, and MM tips refer to tips comprising at least some magnetic material.

The energy delivered by the RF tip to the tissue under treatment may be in the form of an RF field, which heats the tissue under treatment. The volume of tissue treated is related, among other things, to the amount of electromagnetic radiation delivered to the tissue. Possible treatments include, but are not limited to, cutting, thermal lesioning, ablation, cauterization, necrosing and coagulation. RFA may be used to treat all types of body tissues, including, but not limited to, vascular tissue, central and peripheral nervous system tissue, peripheral nerves, tumors, malignant and nonmalignant tissue, and bone. For example, medial branch nerves may be ablated for facet joint denervation, and dorsal rami nerves may be ablated for sacroiliac (SI) and facet denervation. Furthermore, additional treatments include genicular nerve ablation and basil-vertebral nerve ablation.

The thermal lesions created may be precise and strategically located on, in, or around the tissue under treatment. The thermal lesions may be may be small, large, or something in between, depending on the treatment required. The RF tip may be introduced to, or near, the tissue under treatment at a variety of angles relative to the tissue under treatment, and at a variety of depths. For example, the RF tip may be placed adjacent to the nerve at an angle parallel, near-parallel, or adjacent to the long axis of the nerve, e.g., the RF tip does not puncture the nerve, but is positioned at an angle parallel, near-parallel, or adjacent to the flow of nervous information. As another example, the RF tip may be introduced into a nerve tissue at an angle perpendicular to the long axis of the nerve, e.g., the RF tip punctures the nerve at an angle normal to the flow of nervous information.

The RF tips may be positioned in, or adjacent to the tissue under treatment using imaging equipment to aid the medical service provider in locating the exact location to place the RF tip. The medical service provider may use real-time x-ray imaging, and/or CT scanning, ultrasound imaging, or other forms of imaging to provide positional information. If used, the imaging equipment provides real time location information of the RF tip in relation to the tissue under treatment. In embodiments, the RF tips may be positioned using direct visualization techniques. That is, the RF tip may be positioned without the use of use real-time x-ray imaging, and/or CT scanning, ultrasound imaging, or other forms of imaging.

Embodiments described herein take the form of RF tips comprised of one or more magnetic (MM) materials. The MM material may be comprised of at least one of a ferromagnetic material or a ferrite material. The ferromagnetic materials may be comprised of a single MM element, or may be an alloy comprised of more than one MM elements. The ferromagnetic materials may include at least one or more of iron, nickel, cobalt, neodymium, dysprosium, or gadolinium. Furthermore, the ferromagnetic material may be comprised of layered, or multi-layered, materials. The ferromagnetic material may be an elemental, or alloy, material coated with a non-metallic metal or alloy, or non-metal material. Furthermore, the ferromagnetic materials may include at least one or more of pure ferromagnetic metals, ferromagnetic oxides, ferromagnetic nitrides, ferromagnetic sulfides, or ferromagnetic phosphides.

The ferrite material may be selected from among the magnetic ceramic materials. One example is the ferrite comprised of iron (III) oxide (Fe₂O₃), and at least one or more of barium, manganese, nickel, or zinc. The choice of the elements comprising the ferrite material is not limited to iron, barium, manganese, nickel, and zinc, but may include other elements, such as cobalt, manganese, and/or strontium as needed to tailor the desired magnetic and electrical properties of the magnetic electrode tips. In embodiments, the magnetic material may be comprised of a mixture of ferromagnetic and ferrite materials.

The MM tip may be attached to the distal end of the electrode. The attachment of the MM tip to the electrode may be electrically conducting, or electrically insulating. The MM material may be connected to a first electrical circuit, and may have a coil of a conducting material wrapped around the MM tip material in electrical communication with a second electrical circuit.

The MM tip may be in electrical communication with the electrode, which in turn is in electrical communication with the connector cables, which are in turn in electrical communication with the oscillating energy source. The electrode provides a rigid material that can conduct the signals from one or more electrical circuits to the RF tip.

The MM tip may be hollow to allow for delivery of a liquid to the tissue under treatment, or to surrounding tissues. For example, a drug may be delivered directly to the tissue under treatment, or to surrounding tissues. As another example, a saline solution, or other liquid, may be delivered to the tissue under treatment, or to surrounding tissues to wash and/or clean the tissues of blood or debris. Furthermore, a hollow tip may allow for the removal of fluids from the region of the tissue under treatment. For example, suction could be applied through the hollow tip to evacuate fluids, and/or tissues.

The MM tip may be solid. It may be rigid or flexible depending on the type of treatment desired, and/or the type of tissue under treatment. The MM tip may be a stylet that is used without a hollow probe body. Furthermore, the MM tip may be positioned within a hollow probe body.

The MM tip may have a variety of shapes. For example, the MM tip may be generally blunt. A blunt tip may allow the MM tip to be brought into contact with tissue under treatment without puncturing the tissue. Alternatively, the MM tip may have a sharp, needle-like shape. A needle-like shape may, among other things, be capable of penetrating deep into a tissue under treatment to deliver the electromagnetic radiation without unnecessarily damaging surrounding tissues. Furthermore, the MM tip may be generally shaped like a knife blade and may, among other things, be capable of cutting tissue in addition to delivering electromagnetic radiation.

The MM tip may have a variety of sizes. For example, an MM tip may have a tip diameter from between about 27 gauge to about 12 gauge. Furthermore, MM tip lengths may vary in length from about 0.5 mm to about 50 mm. These descriptions of sizes are not intended to be exclusive, but are intended to reflect typical size ranges. As treatment needs vary, so may tip diameters and lengths vary beyond the ranges described.

The MM tip may be coated with a metal, a non-metal, or any combination of metal and non-metal. A coating on the MM tip may be a non-magnetic metal, including at least one or more of aluminum, copper, lead, nickel, tin, titanium, zinc, niobium, tantalum, vanadium, gold, silver, or palladium. A coating on the MM tip may be a non-metal material, and may include one or more of a plastic, a polymeric material, or a composite material.

A coating on the MM tip may be formed from a dispersion, and/or a mixture of metal particles mixed into a metal, or a non-metal. The metal particles in the dispersion may be in the form of powders, flakes, or grains, and may vary in size from many microns to nano particles. For example, MM nanoparticles may be dispersed in a non-magnetic metal. As another example, MM nanoparticles may be dispersed in a non-metal material. The nanoparticles may be in size between about 0.1 nm to about 1000 nm.

Furthermore, a coating on the MM tip may confer any one of many advantages over an uncoated MM tip. For example, a coating on the MM tip may protect the MM tip from potentially corrosive fluids and tissues in the patient. As another example, a coating may be applied to the MM tip to reduce drag on movement of the RFA probe as the MM tip is directed within the body of the patient by a medical service provider.

The electrode may be positioned within a hollow probe body. The hollow probe body may be electrically isolated from one or more of the electrode, the MM tip, and the electrical connectors. The hollow probe body may be fashioned from one or more of a plastic, a polymeric material, a composite material, or a metal. Generally, the hollow probe body is configured to be held comfortably in the hand of a medical service provider, and may also be designed to have an aesthetically pleasing appearance. Wires, cables, or other conducting means may be distributed within the hollow probe body to conduct electromagnetic radiation, and/or sensor readings.

The hollow probe body may have a sharp point at the distal end that allows for penetrating tissue. In some medical treatment situations, it may be necessary for the medical service provider to use the sharp end of the hollow probe body to penetrate tissue, such as bone, cartilage, or connective tissue. For example, a sharp end of the hollow probe body may be used to penetrate a vertebral body so that treatment may occur in the bone marrow of the vertebral body.

The hollow probe body may have markings on the outside of the body that may be used to determine depth of penetration of the RFA probe into the patient being treated. The markings may be scored into the probe body, may be applied by a printing or labeling process, or may be some combination of the two.

The RF tip may be comprised of one or more tines. The tines may be on a single circuit, or may be on individual circuits. The more than one tines may be positioned such that each one has the same length, and together the tips of the tines define a plane normal to the direction of the electrode. Alternatively, the tines may have different lengths. The lengths of the tines may be adjustable so that the tissue under treatment may be exposed to an optimal RF field for the desired outcome.

In embodiments, the RF tip may have a portion of the tip at the most distal end that is not energized. In other words, the tine may be energized to produce electromagnetic radiation down a length of the electrode conducting portion without going to very end. A metal, a non-metal, or any combination of metal and non-metal metal may be positioned at the most distal end of the tip to provide an “inert” portion at the very end of the tip. In this position, a metal, a non-metal, or any combination of metal and non-metal metal may be an inert material, meaning that it provides little or no heating to the tissue under treatment. The presence of the inert material may reduce unintended damage to tissue adjacent to the tissue under treatment.

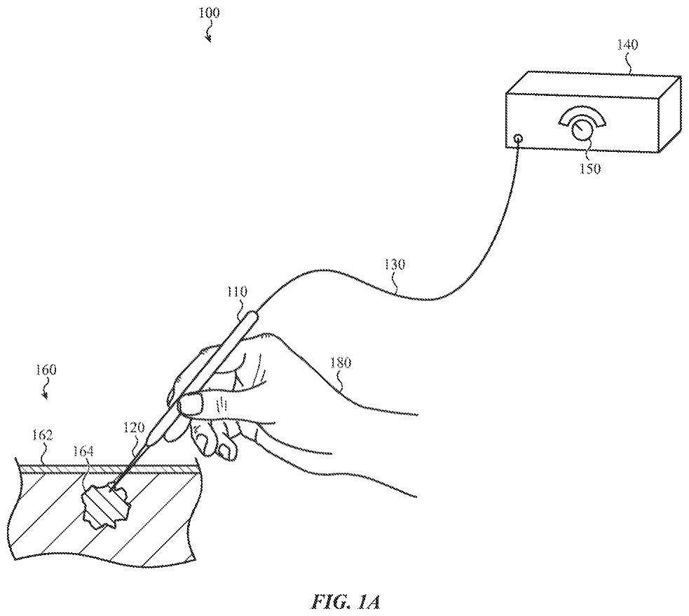

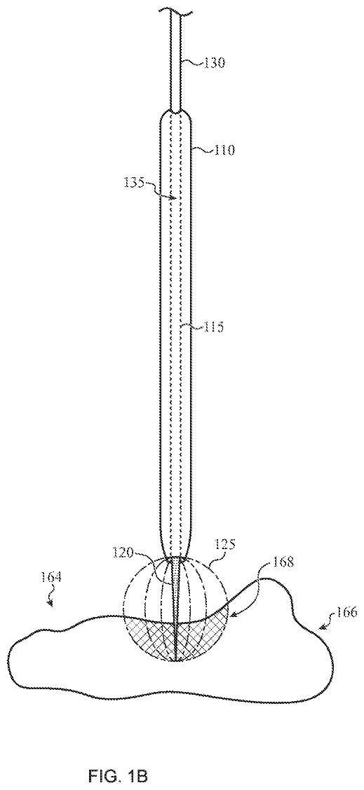

These and other embodiments are discussed below with reference to FIGS. 1A-7B. However, those skilled in the art will readily appreciate that the detailed description given herein with respect to these figures is for explanatory purposes only and should not be construed as limiting.

FIG. 1A is a system diagram illustrating certain components of an RFA system 100, in accordance with embodiments described herein. In particular, FIG. 1A illustrates a user interacting with a sample RFA system and a patient showing aspects of a sample embodiment. The RFA system 100 includes an RFA probe 110 having an RF tip 120 at a distal end of the RFA probe 110, and connector cables 130 in electrical communication to the proximal end of the RFA probe 110 providing electrical communication to a control unit/an oscillating energy source 140.

The RFA system 100 may be used to treat a medical patient 160. The RFA system 100 may be configured to permit a user 180 to manipulate the RFA probe 110 to penetrate the skin of a patient 162 to insert the RF tip 120 into a tissue under treatment 164. The user 180, typically a medical service provider, may additionally or alternatively position the RF tip 120 adjacent to the tissue under treatment 164.

An embodiment of the invention is provided in FIG. 1B showing a cross section view of an RFA probe 110 inserted into a tissue under treatment 164. The RFA probe 110 comprises an electrode conducting portion 135 positioned within a hollow probe body 115. An RF tip 120 is positioned at the distal end of the electrode conducting portion 135, and connector cables 130 are in electrical communication with the electrode conducting portion 135. The electrical connector cables 130 are in electrical communication with an oscillating energy source 140.

The medical treatment performed on the tissue under treatment 164 is accomplished by an oscillating RF field generated at the RF tip 120 from electromagnetic radiation produced by the oscillating energy source 140. The RF field is represented by RF field lines 125 schematically illustrated in FIG. 1B. As illustrated in FIG. 1B, the RF field defines a treatment volume of electromagnetic radiation (illustrated by the RF field lines 125) that can be applied to a tissue under treatment 164. After application of electromagnetic radiation to the tissue under treatment 164, the tissue under treatment 164 includes a region of treated tissue 168 and a region of untreated tissue 166.

The treatment volume of electromagnetic radiation generated is, among other things, related to power of the RF signal transmitted to the RF tip. The power of the electromagnetic radiation produced within the oscillating energy source 140 can be adjusted using a tuner 150 (FIG. 1A) located on the oscillating energy source 140. In embodiments, the electromagnetic radiation produced within the oscillating energy source 140 can be adjusted using a tuner (not shown) located at a distance from the oscillating energy source 140. As one example, this tuner may be located on the RF ablation probe 110 (not shown) and controlled by pushing with a finger. As another example, a tuner (not shown) may be located in a foot pedal that is controlled by foot movements of the medical service provider, e.g., a user. In embodiments, the tuner may be controlled by a timer, such that a set power may be delivered for a duration that is preselected, and then measured by a timing mechanism that turns off the power at the end preselected period of time.

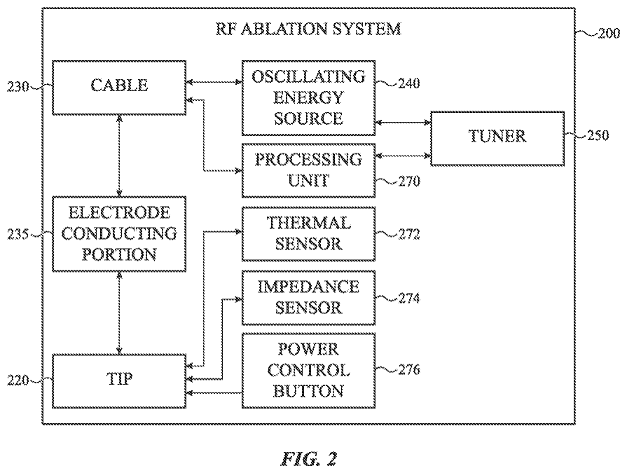

FIG. 2 depicts a simplified block diagram of an embodiment of an RFA system 200 including an RF tip and various other portions thereof. For simplicity of illustration, the system diagram may be presented without signal and/or interconnection paths between system elements that may be required or desirable for a particular embodiment. Accordingly, the absence or presence of a signal path and/or interconnection path between various system elements of the simplified system diagrams depicted in FIG. 2 is not construed as a preference or requirement for the presence or absence of any particular electrical or mechanical relationship between the various system elements.

Reference is made to operational components of the RFA system 200 depicted in FIG. 2, as well as FIGS. 1A and 1B. The RFA system 200 includes an RF tip 220 that is in electrical communication with an electrode conducting portion 235, a connector cable 230, and an oscillating energy source 240. The oscillating energy source 240 provides electromagnetic radiation for treating tissues. The electromagnetic radiation is conducted through the connector cable 230 to the electrode conducting portion 235, and electromagnetic radiation is radiated out of the RF tip 220 as an RF field. (The RF field is depicted in FIG. 1B as RF field lines 125.) The electrode conducting portion 235 is located within an RFA probe 110 as pictured in FIGS. 1A and 1B, and the RF tip 220 is extended beyond the distal end of the RFA probe 110 to facilitate contact with the tissue under treatment 164.

The attachment of the RF tip 220 to the electrode conducting portion 235 is both mechanical and electrical. The mechanical connection between the RF tip 220 and the electrode conducting portion 235 is rigid and capable of remaining intact as the RF tip 220 is inserted into the tissue under treatment 164. The RF tip 220 is also in electrical communication with the electrode conducting portion 235. The electrical connection between the RF tip 220 and the electrode conducting portion 235 may include one or more electrical circuits, depending on the type and configuration of the RF tip 220. Various types and configurations of the RF tip 220 will be discussed below.

The connector cable 230 provides a flexible, adjustable electrical connection between the oscillating energy source 240 and the electrode conducting portion 235. The connector cable 230 may actually contain more than one isolated transmission line within a single external insulating sheath. The connector cable 230 may be electrically coupled with the oscillating energy source 240 and the electrode connecting portion 235 by any variety of connectors and/or plugs.

The amount of electromagnetic radiation delivered by the RF tip 220 is, in part, determined by the amount of energy generated by the oscillating energy source 240. A tuner 250 is configured to control the amount of RF power delivered to the RF tip 220. The tuner 250 is also in electrical communication with a processing unit 270. The tuner 250 may be provided on the oscillating energy source 240 as illustrated in FIG. 1A. (In FIG. 1A, the tuner is shown on the exterior of the oscillating energy source 140.) Furthermore, a remote tuner 250 (not shown) may be in electrical or wireless communication to the processing unit 270. The remote tuner 250 may be controlled by a foot of the medical service provider to allow hands-free tuning of the RF power.

The processing unit 270 controls the energy output of the oscillating energy source 240, and may receive sensor input from the RF tip 220, e.g., the processing unit 270 may be configured to provide feedback control of the RF ablation process. In addition to receiving user input through the tuner 250, the processing unit 270 also receives sensor and control input conducted through the RFA probe 110 from the RF tip 220. At least one thermal sensor 272 and at least one impedance sensor 274 provide sensor output that is carried back to the processing unit 270.

The one or more thermal sensors 272 detect the temperature of the tissue under treatment 164 and provide that information to the processing unit 270. The temperature of the tissue under treatment 164 provides information to the medical service provider of the extent of ablation that is occurring with the treatment. The processing unit 270 may use the changes in the temperature of the tissue under treatment 164 in controlling the power output of the oscillating energy source 240.

The one or more impedance sensors 274 detect the changes in how the tissue under treatment is responding to the electromagnetic radiation being provided through the RF tip 220. The processing unit 270 may use the changes in the impedance in controlling the power output of the oscillating energy source 240.

Optionally, the RF ablation system 200 may incorporate a power control button 276 on the RFA probe. The medical service provider may use the power control button 276 to raise or lower the electromagnetic radiation being sent to the RF tip 220. The input from the power control button 276 is transmitted to the processing unit 270. The signal from the power control button 276 may be carried through the electrode conducting portion 235, or through electrical conductors distributed between the electrode conducting portion 235 and the RFA probe, to the connector cable 230, in an arrangement similar to how the outputs from the one or more thermal sensors 272 and one or more impedance sensors 274 are sent to the processing unit 270.

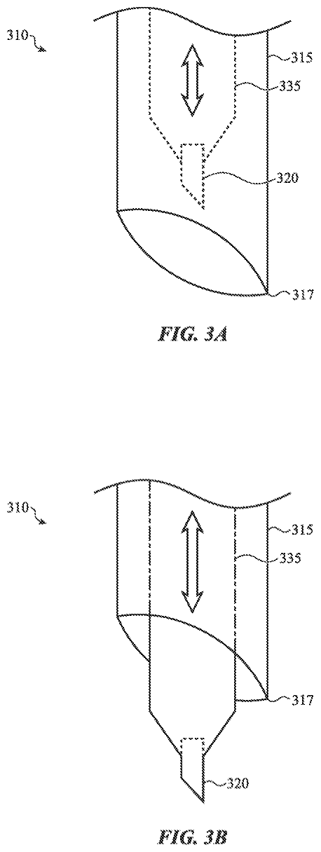

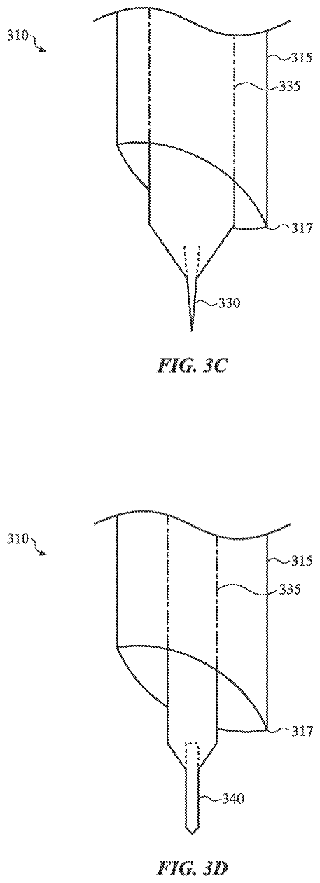

FIGS. 3A and 3B illustrate embodiments of a cut away portion of an RFA probe 310, showing an RF tip 320 connected to an electrode conducting portion 335 and positioned within a hollow probe body 315. In FIG. 3A, the electrode conducting portion 335 is retracted within the hollow probe body 315. In this embodiment, the hollow probe body 315 has a sharpened point 317 at the distal end of the hollow probe body 315 that can be used to penetrate hard tissue, such as bone, cartilage, and connective tissues. In an alternative embodiment, the distal end of the hollow probe body 315 may be blunt.

Once the hard tissues have been penetrated, the RF tip 320 at the distal end electrode conducting portion 335 may be extended out of the hollow probe body 315, as illustrated in FIG. 3B. This arrangement is beneficial, for example, when performing basivertebral nerve ablation. In this procedure, the sharpened point 317 of the hollow probe body 315 is forced through the boney exterior structure of a vertebral body so that the RF tip 320 can be extended out of the hollow probe body 315 through the opening and into the vertebral body into the basivertebral nerve. Once the RF tip 320 is positioned in, or adjacent to, the basivertebral nerve, it can be ablated by application of electromagnetic radiation through the RF tip 320.

FIGS. 3C and 3D illustrate embodiments of different shapes of the RF tips. As shown in FIG. 3C, the RF tip 330 may be needle-like and narrow, forming a sharp point. This shape may be particularly useful in delivering the RF tip 330 deep into a tissue under treatment and minimizing damage to surrounding tissue. Another example of a potential shape of an RF tip is shown in FIG. 3D. The RF tip 340 illustrated in FIG. 3D is generally blunt. A blunt RF tip 340 may also be used to treat tissue without puncturing the tissue.

In embodiments, the RF tip 120 of the RF ablation system 100 may be formed from magnetic (MM) material. The use of an MM material in the RF tip 120 has advantages over RF tips 120 made from non-MM material. For example, MM material may improve the targeting of tissues for treatment. In particular, the use of MM material may improve control over shaping the RF field generated at the RF tip 120. In other words, by incorporating MM material into the RF tips 120 used in an RFA system 100, the safety and efficacy of RFA treatments may be improved.

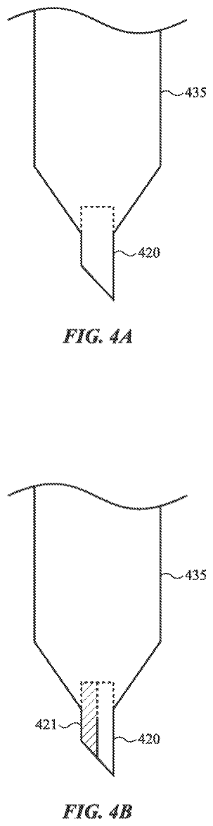

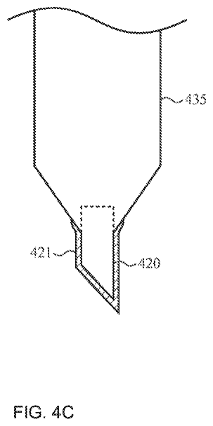

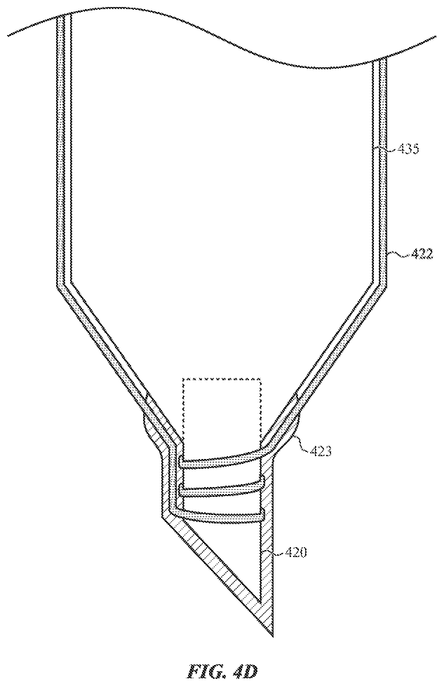

FIG. 4A illustrates an embodiment of an RF tip positioned on an electrode conducting portion 435. In this embodiment, the RF tip is formed from an MM material to make an MM tip 420. The MM material may be comprised of a single MM element, such as iron, nickel, cobalt, neodymium, dysprosium, or gadolinium. The selection of the MM element may be influenced by the desired magnetic field strength, manufacturability, and cost, among other factors.

In another embodiment, the MM tip 420 may be comprised of an alloy of MM elements, including one or more of iron, nickel, cobalt, neodymium, dysprosium, or gadolinium. A MM alloy may include Permalloy, an alloy comprised of iron and nickel, as well as alloys of iron and cobalt.

In yet another embodiment, the magnetic materials may include at least one or more of MM oxides, MM nitrides, MM sulfides, or MM phosphides. Magnetite, an oxidized iron material, is an example of a MM oxide.

In yet another embodiment, the MM tip 420 material may be comprised of alloys of magnetic materials interspersed with non-magnetic materials. The alloys may be designed to increase or decrease the magnetic field strength of the MM material. Non-magnetic materials may be metals, including one or more of boron, aluminum, copper, lead, nickel, tin, titanium, zinc, niobium, tantalum, vanadium, gold, silver, or palladium. In an embodiment, an alloy MM tip 420 may be made from neodymium, iron, and boron, known as a neodymium magnet. The alloys may be comprised of dispersions (e.g., mixtures) of nanoparticles of the MM metals in the non-MM metal materials. The nano particles may be in size between about 0.1 nm to about 1000 nm.

In yet another embodiment, the MM tip 420 material may be comprised of layered, or multi-layered, materials. The layered and multilayered materials may include layers of magnetic materials, or layers of magnetic materials interspersed with non-magnetic materials.

FIG. 4B illustrates an embodiment of an MM tip 420 having a portion of a non-magnetic material 421 on the MM tip 420. The portion 421 may be a non-MM metal, including one or more of aluminum, copper, lead, nickel, tin, titanium, zinc, niobium, tantalum, vanadium, gold, silver, or palladium. The non-magnetic material 421 may be adjacent to the magnetic material on the MM tip 420. The magnetic material may be at the point of the MM tip 420 and attached to the electrode conducting portion 435. The non-magnetic material 421 on the MM tip 420 may increase thermal control of the MM tip 420 and reduce the risk of injuring untreated tissue outside of the target tissue under treatment. The thickness of non-magnetic material 421 may facilitate either increasing or decreasing thermal conductivity based on the specific medical treatment for which the MM tip 420 is designed. Furthermore, the MM tip 420 may be shaped with a sharp edge on either, or both sides of MM tip 420. The sharp edge may be used to cut and or penetrate the tissue under treatment.

FIG. 4C illustrates an embodiment of an MM tip 420 having a casing of a non-magnetic material 421 on the MM tip 420. The casing may be a non-MM metal, including one or more of aluminum, copper, lead, nickel, tin, titanium, zinc, niobium, tantalum, vanadium, gold, silver, or palladium. The casing may fully surround the MM tip 420 and attach to the electrode conducting portion 435. The non-magnetic material 421 on the MM tip 420 may increase thermal control of the MM tip 420 and reduce the risk of injuring untreated tissue 166 outside of the target tissue under treatment. The thickness of non-magnetic material 421 may be optimized to either increase or decrease thermal conductivity based on the specific medical treatment for which the MM tip 420 is designed.

Another embodiment of a casing of a non-magnetic material 421 on an MM tip 420 is that the non-magnetic material 421 may be a non-metal material. The non-MM casing material may include one or more of a plastic, a polymeric material, or a composite material. An advantage of having a non-metal as the non-magnetic material 421 on the MM tip 420 is that a non-magnetic material 421 may be electrically insulating. Having an electrically insulating casing 421 on the MM tip 420 may be advantageous in applications of RFA close to metal inserts or parts inside the patient that are in close proximity to the tissue under treatment 164.

FIG. 4D illustrates an embodiment of an MM tip 420 with a non-MM casing 423 having a coil 422 of a conducting material wrapping around the MM tip 420 and the non-MM casing 423. In one embodiment, the non-MM casing 423 may be a non-MM metal, including one or more of aluminum, copper, lead, nickel, tin, titanium, zinc, niobium, tantalum, vanadium, gold, silver, or palladium. The casing fully surrounds the MM tip 420, fully encases conducting coil 422, and attaches to the electrode conducting portion 435. In this embodiment, the conducting coil 422 is coated in an electrically insulating material so that the MM tip 420 is electrically isolated from the conducting coil 422. Furthermore, the MM tip 420 may be connected to a first electrical circuit, while the conducting coil 422 may be connected to a second electrical circuit. In this embodiment, the conducting coil 422 may be used to enhance the magnetic field strength of the MM tip 420.

In another embodiment, the MM tip 420 may have a non-MM casing 423 made from a non-metal material surrounding the MM tip 420 and encasing the conducting coil 422. In this embodiment, the non-MM casing 423 material may include one or more of a plastic, a polymeric material, or a composite material. In this embodiment, the conducting coil 422 may be coated with an electrically insulating material, or it may not. Since the non-MM casing is electrically insulating, the casing provides electrical isolation between the MM tip 420 and the conducting coil 422. This embodiment provides enhanced magnetic field strength of the MM tip 420 combined with an electrical insulator around the MM tip 420 for applications of RFA close to metal inserts or parts inside the patient that are in close proximity to the tissue under treatment 164.

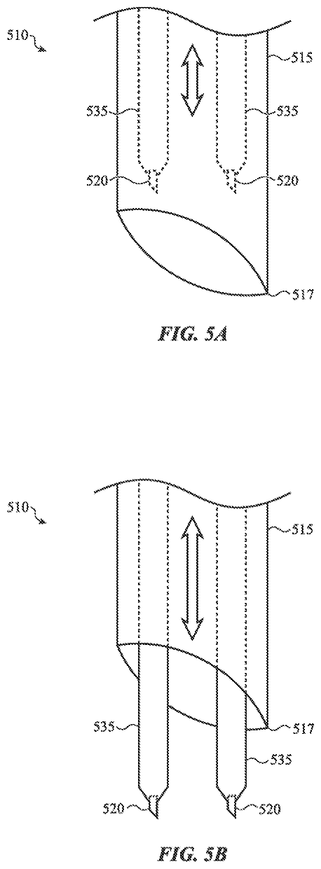

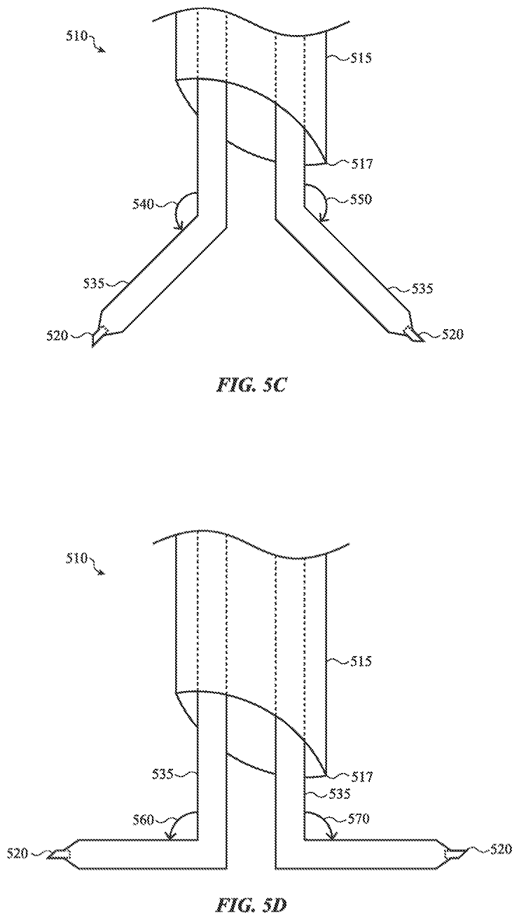

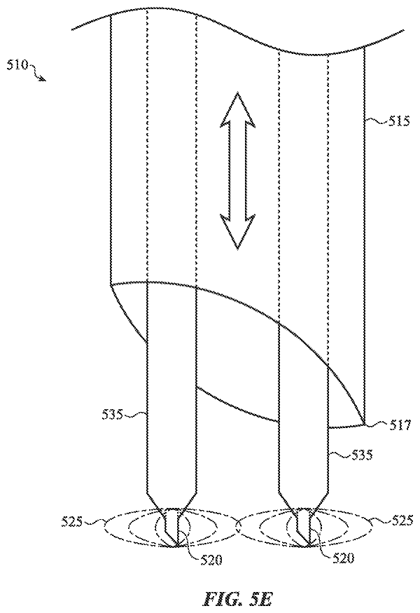

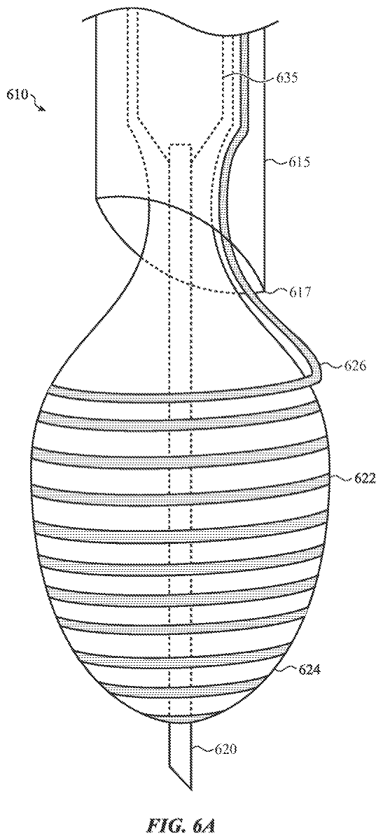

FIGS. 5A-5E generally illustrate an RFA ablation probe having two tines with MM tips. FIG. 5A includes two electrode conducting portions 535, each with MM tips 520, representing two tines. The two tines are retracted into the hollow probe body 515. The hollow probe body has a sharp point 517 for penetrating hard tissue such as bone, cartilage, and connective tissue. FIG. 5B illustrates the tines being extended out of the hollow probe body 515.

FIGS. 5C and 5D illustrate RFA ablation probes that have bends in the tines. FIG. 5C illustrates tines which have angled bends 540, 550 in the left tine and right tine respectively. As shown in FIG. 5C, angles 540, 550 are approximately 45 degrees, and are approximately equal. However, in embodiments, the angles of the tines may vary from 45 degrees, and may vary from each other (not shown). Furthermore, in embodiments, the tines may be pointing away from each other, as illustrated in FIG. 5D. In FIG. 5D, the angles 560, 570 are approximately 90 degrees, and are approximately equal. Further, in some embodiments, one or both angles 560, 570 may vary from 90 degrees, and may vary from each other (not shown).

In FIG. 5E, the RF fields generated by the MM tips 520, positioned on the electrode conducting portions 535, are schematically illustrated by RF field lines 525. As illustrated in FIG. 5E, the RF field defines a treatment volume of electromagnetic radiation (illustrated by the RF field lines 525) that can be applied to a tissue under treatment. In this embodiment, the two tines potentially provide an application of overlapping Electromagnetic radiation to a tissue under treatment (not shown).

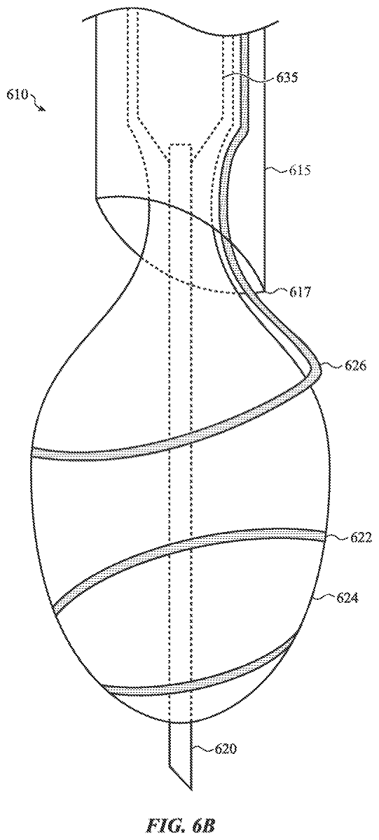

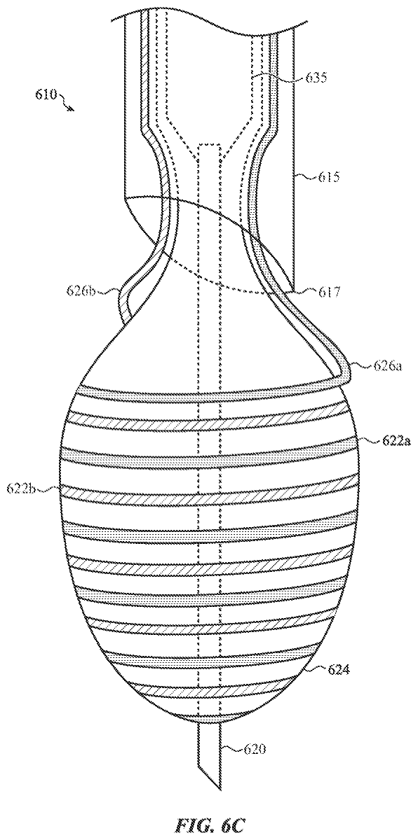

FIG. 6A illustrates an RFA ablation probe having a balloon expander for large volume treatment. An MM tip 620 is attached to an electrode conducting portion 635. The hollow probe body 615 has a sharp point 617 at the distal end. An expandable balloon 624 surrounds the electrode conducting portion 635 and a portion of the MM tip 620. The expandable balloon 624 has a conducting coil 622 wrapping around the expandable balloon 624 with electrical leads 626 running up the hollow probe body 615 next to the electrode conducting portion 635.

In an embodiment, the MM tip 620 may be in electrical communication with a first electrical circuit, and the electrical leads 626 may be in electrical communication with a second electrical circuit.

In another embodiment, the MM tip 620 and the expandable balloon 624 with the conducting coil 622 may be retracted into the hollow probe body 615 so that the sharp point 617 of the hollow probe body 615 may be inserted through hard tissue into a treatment volume into which the expandable balloon 624 may be deployed. The conducting coil 622 is flexible so that it can conform to the shape of expandable balloon 624 whether the expandable balloon 624 is collapsed and retracted into the hollow probe body 615, or is fully expanded. Once deployed, an RFA may be applied to the tissue under treatment.

In some embodiments, the conducting coil 622 may be distributed around the expandable balloon in a variety of patterns. FIG. 6B illustrates another embodiment of an RFA ablation probe having a balloon expander for large volume treatment. In FIG. 6B, the conducting coil 622 has an alternate pattern, and so wraps fewer times around the expandable balloon.

In certain embodiments, an expandable balloon 624 may have more than one conducting coil electrically connected to more than one electrical circuit. FIG. 6C illustrates an expandable balloon 624 having two conducting coils. FIG. 6C illustrates a first conducting coil 622a that may be connected to a first electrical circuit by first electrical leads 626a, and a second conducting coil 622b that may be connected to a second electrical circuit by second electrical leads 626b. Furthermore, the MM tip may be connected to a third electrical circuit by the electrode conducting portion 635. The first, second, and third electrical circuits may be energized at the same power levels, or may be energized at different power levels. For example, the medical service provider may treat a tissue with only the MM tip and one conducting coil energized, and then energize the second conducting coil for delivering additional Electromagnetic radiation. In embodiments, any combination of energizing the various electrical circuits is contemplated.

In certain embodiments, an expandable balloon 624 may be configured to provide or otherwise operate as an RFA probe in either a monopolar or bipolar mode. For example, in bipolar mode, one lead may be connected to a ground lead and another lead to an RF lead. FIG. 6C illustrates an expandable balloon 624 having two conducting coils, each attached to (or incorporated with) a separate lead. In the sample embodiment of FIG. 6C, a first conducting coil 622a may be connected to a ground lead 626a, and a second conducting coil 622b may be connected to a radio frequency lead 626b. In other embodiments, both coils 622a, 622b may be connected to the same lead.

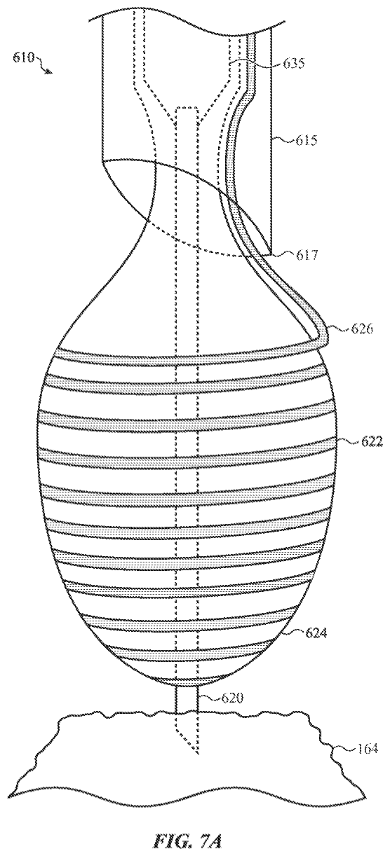

FIGS. 7A and 7B illustrate an embodiment of a potential use of an expandable RFA ablation probe having a balloon expander. FIG. 7A illustrates an expandable balloon prior to the balloon physically engaging with a tissue under treatment. The MM tip 620 is engaged with the tissue under treatment 164 prior to engagement of the balloon expander 624.

FIG. 7B illustrates an embodiment of an RFA probe with an RFA tip and an expandable balloon after the balloon has physically engaged with a tissue under treatment. The expandable balloon 624 has conformed to the shape of the tissue under treatment 164 allowing the flexible conducting coil 622 to deliver electromagnetic radiation directly to the tissue under treatment 164.

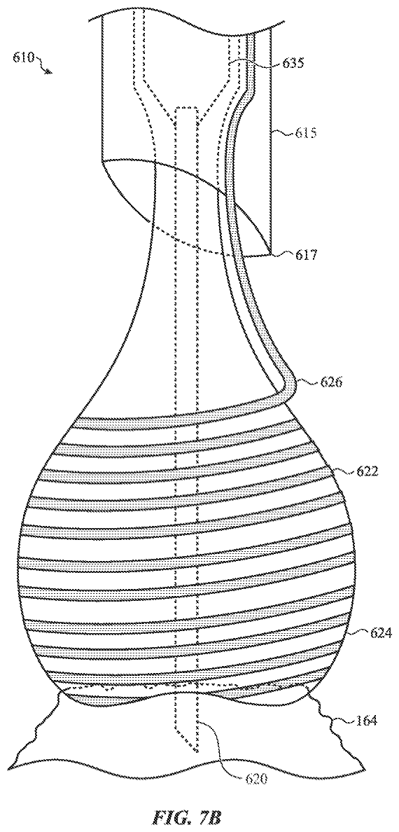

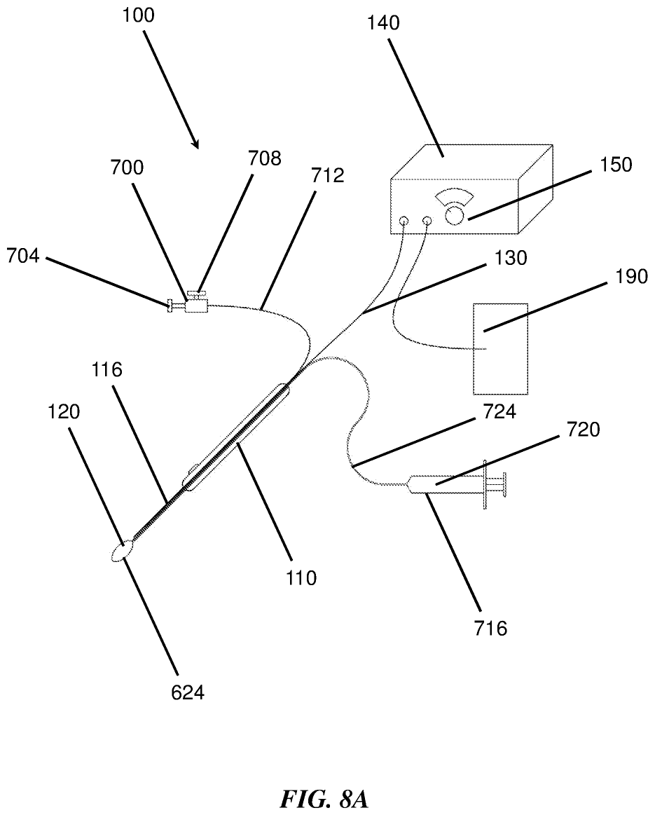

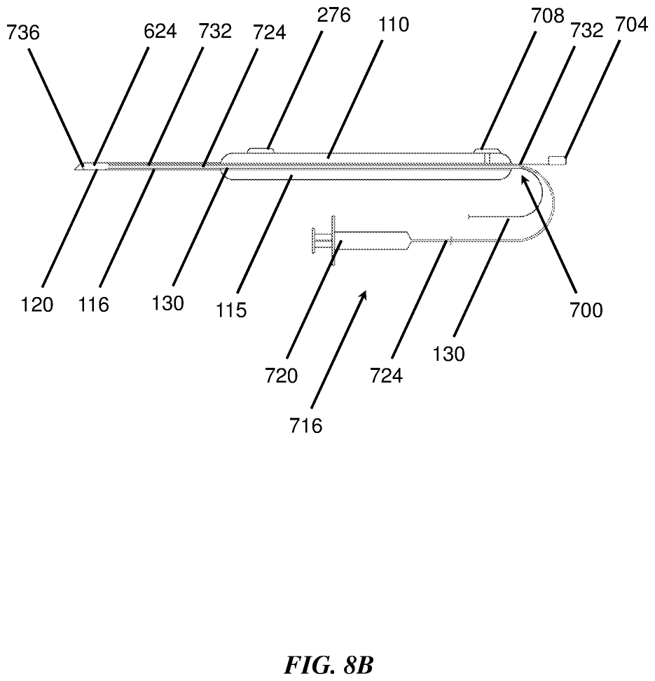

FIG. 8A shows another system diagram illustrating an RFA system 100. The RFA system 100 includes an RFA probe 110 having an RF tip 120 at a distal end of the RFA probe 110, and connector cables 130 in electrical communication to the proximal end of the RFA probe 110 providing electrical communication to an oscillating energy source 140. The RFA system 100 may be configured to permit a user 180 to manipulate the RFA probe 110 to penetrate tissue of a patient 162 to insert the RF tip 120 into a tissue under treatment 164. The medical treatment performed on the tissue under treatment 164 is accomplished by an oscillating RF field generated at the RF tip 120 from electromagnetic radiation produced by the oscillating energy source 140. The RFA system may also include a ground pad 190 for use with monopolar treatment. The oscillating energy source typically operates at a frequency between about 0.3 MHz and about 10 MHz. The RFA system typically includes the various electrical components and other components as are described in FIG. 2.

In many examples, the probe 110 may include a retractable tip 120 and the tip 120 may include an expandable balloon 624 to provide a treatment area with a desired shape and volume. The probe 110 and system 100 may include a retractor 700 which is operable to extend and retract the probe tip 120 from within a hollow probe body member 115. Often, the probe body includes a cannula 116 or hollow needle which is sufficiently large to hold the probe tip 120 in the lumen thereof and sufficiently small to allow a user to place the probe tip 120 in a desired body tissue. The retractor 700 may include a movable finger lever or plunger 704 which is manually movable by a person using the probe 110 to extend or retract the probe tip 120. The retractor 700 may also include a lock 708 such as a latch, push button lock, or thumb screw which is selectively lockable to fix the finger lever or plunger 704 in place and thereby lock the probe tip 120 in a particular state of extension or retraction. The retractor 700 may be attached to the body of the probe 110, such as by having the retractor 700, finger lever/plunger 704, and lock 708 attached to the proximal end of the body of the probe 110. Alternatively, the retractor 700 may be separated from the body of the probe 110 by a retractor extension 712. The finger lever or plunger 704 may be connected to the probe tip 120 by a flexible rod or a braided cable which may be made of metal, polymeric, or composite material. Accordingly, the extension 712 may include a length of the flexible rod inside of a sheath.

The system 100 may also include a fluid pump 716 which is connected to the probe tip (e.g. the expandable balloon) via a fluid channel and which is operable to pump fluid (such as saline or air) into and out of the expandable balloon 624. In a simple embodiment, the fluid pump 716 may be a syringe 720 which is attached to the expandable balloon 624 by a length of tubing 724. The fluid pump 720 may alternately use a chamber and piston driven by a threaded rod, a small collapsible bulb, or another arrangement to pump fluid into the expandable balloon 624. The fluid pump 716 may be located on or near the probe 110 or may be separated from the body of the probe 110 by a length of tubing 724 depending on size and maneuverability requirements for the probe 110.

FIG. 8B shows a drawing of another example RFA probe 110. The probe includes a hollow probe body 115 with a passage extending through the probe body 115. A cannula 116 extends distally from the distal end of the probe body 115. The cannula 116 includes a lumen which is in communication with the probe body 115. An expandable probe tip 120 is disposed inside of the cannula lumen near the distal end of the cannula 116. The expandable probe tip 120 is connected to a retractor 700. More particularly, the expandable probe tip 120 is connected to a retraction rod 732 which is in turn connected to a retraction lever/plunger 704. The retraction lever 704 and retraction rod 732 may be used to move the probe tip 120 distally or proximally relative to the cannula 116 in order to deploy the probe tip 120 from the cannula 116 or to retract the probe tip into the lumen of the cannula. A lock 708 allows the retraction rod 732 to be locked in a desired position to prevent distal or proximal movement of the probe tip 120 relative to the cannula 116. As discussed in greater detail below, the expandable probe tip 120 includes a balloon 624 which may be expanded or contracted to a desired size for treatment.

The balloon 624 is connected to a fluid pump 716 via tubing 724. The fluid pump 716 may be simple such as a syringe 720 or may be an electronically controlled pump, etc. The fluid pump 716 introduces fluid into the deployed expandable tip 120 in order to expand the tip 120 and withdraws fluid from the expandable tip 120 to collapse the tip. The tubing 724 may slide distally and proximally through the hollow probe body 115 as the probe tip 120 is deployed from the probe body or retracted into the probe body. The probe tip 120 includes one or more electrode wires 736 forming an active treatment region 750 on part or all of the surface of the expandable probe tip 120. The active treatment region 750 on the probe tip 120 is energized by the power supply 140 and emits energy into a target tissue 164 for treatment. The electrode wires 736 are connected to the energy source 140 by electrical connector cables 130. The probe 110 may also include a user control interface such as a power control button 276 which may be used to control the energy level of the probe tip 120 or energize the probe tip. Distance between the probe 110 and the retractor 700, the fluid pump 716, and the power source 140 may vary.

For many of the expandable probe tips 120 disclosed herein, the probe tip 120 is a generally ellipsoid shape. The probe tip 120 has a first, collapsed state and a second, expanded state. In the first collapsed state, the probe tip 120 is generally elongate and has a small diameter. In this first collapsed state the probe tip 120 fits within the lumen of the cannula 116 and can slide out of and into the cannula for deployment or retraction. In the first collapsed state, the probe tip 120 may be viewed as largely cylindrical due to its length and small diameter relative to its length. The probe tip may often include a diameter which is between about one half and about one eighth of its length. For many embodiments of the probe tip, the diameter of the probe tip 120 in the collapsed state may be about one third or about one fourth of its length. In the second expanded state the probe tip 120 is larger in diameter and often somewhat shorter in length than in the first collapsed state. In the second state, the probe tip 120 typically provides a larger surface area and allows for treatment of a larger area or volume of target tissue. In the second, expanded state the probe tip 120 is more oval or egg shaped in appearance and often has a diameter which is approximately equal to its length or about one half or one third of its length.

In one example probe 110, the cannula 116 may be formed from a rigid material such as stainless steel or titanium. This cannula provides sufficient strength to penetrate tissue such as bone. This cannula 116 also allows for medical imaging techniques to be used to visualize the placement of the cannula. The cannula 116 may often be between about 0.1 inches in diameter and about 0.2 inches in diameter. The cannula 116 is often between about 2 inches long and about 6 inches long. In another example probe 110, the cannula may be formed of a material such as a plastic material. The size of the expandable probe tip 120 may be selected according to the target tissue being treated. A variety of probes 110 with different sizes and configurations of probe tips 120 may be available to allow a medical practitioner to select a probe 110 with a probe tip 120 most suited to the target tissue. Expanded probe tips 120 may often be between about 0.1 inches in diameter and about 0.7 inches in diameter, and are often between about 0.2 inches and in diameter and about 0.5 inches in diameter. The expanded probe tips 120 are often between about 0.2 inches long and about 1 inch long. More particularly, the expanded probe tips are often between about 0.5 inches long and about 0.8 inches long.

Probe tips 120 with active treatment regions 750 on the distal or proximal ends of the probe tip 120 are often more spherical in shape. Accordingly, these expanded probe tips 120 may often be between about 0.2 inches in diameter and 0.2 inches in length and about 0.5 inches in diameter and 0.5 inches in length.

Probe tips with active treatment regions on a side of the probe tip 120 are often more elongate in shape. Accordingly, these expanded probe tips 120 may often be between about 0.2 inches in diameter and about 0.5 inches in diameter and between about 0.2 inches in length and about 1 inch in length. Such a probe tip 120 which is about 0.2 inches in diameter is often between about 0.3 inches in length and about 0.5 inches in length. Such a probe tip 120 which is about 0.5 inches in diameter is often between about 0.8 inches in length and about 1 inch in length.

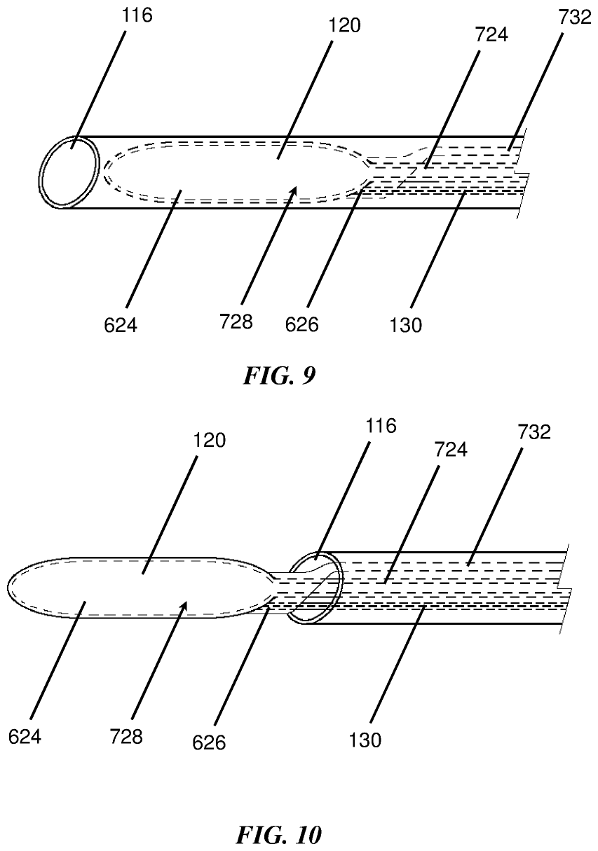

FIGS. 9 through 11 show deployment of a probe tip 120 for use in treating a target tissue. For clarity, only the distal end of the probe 110 is shown. FIG. 9 shows the distal end of the cannula 116 with the probe tip 120 retracted within the lumen of the cannula 116. The probe tip 120 includes an expandable balloon 624. The expandable balloon 624 includes a hollow cavity 728 which may receive fluid to expand the balloon 624. The balloon may include RF electrode wires 736 disposed around the balloon and connected to electrical leads 626 as discussed with respect to the other figures herein for treatment purposes. These wires are not shown around the balloon 624 to facilitate understanding of the deployment of the balloon 624 and probe tip 120. The proximal end of the expandable balloon 624 is connected to the tube 724 such that fluid may flow between the lumen of the tube 724 and the interior cavity of the expandable balloon 624. The proximal end of the expandable balloon 624 is also connected to a retraction rod 732 which is itself connected to the retractor 700 such that movement of the finger lever/plunger 704 moves the advance rod and thereby moves the expandable balloon 624/probe tip 120 proximally or distally relative to the cannula 116.

During a first portion of a surgical procedure, probe 110 is in the configuration shown in FIG. 9 with the retractable balloon 624/probe tip 120 disposed in the distal end of the cannula 116. The distal end of the cannula 116 is then introduced into a patient's body so that the distal end of the cannula 116 is placed in a desired location relative to a target tissue to be treated. As is shown in FIG. 10, the lock 708 is unlocked and the finger lever/plunger 704 is then moved to extend the expandable balloon 624/probe tip 120 from the cannula 116 and thereby deploy the probe tip 120/retractable balloon 624. The finger lever moves the retraction rod 732 and thereby moves the probe tip 120 and expandable balloon 624. Once the expandable balloon 624 is extended a desired amount from the cannula 116, the lock 708 may be engaged to prevent movement of the probe tip 120 and expandable balloon 624 relative to the cannula 116. In many example probes 110, the retractor rod 732, tubing 724, and electrical leads 626 move together within the lumen of the cannula 116 to deploy or retract the probe tip 120 and expandable balloon 624.

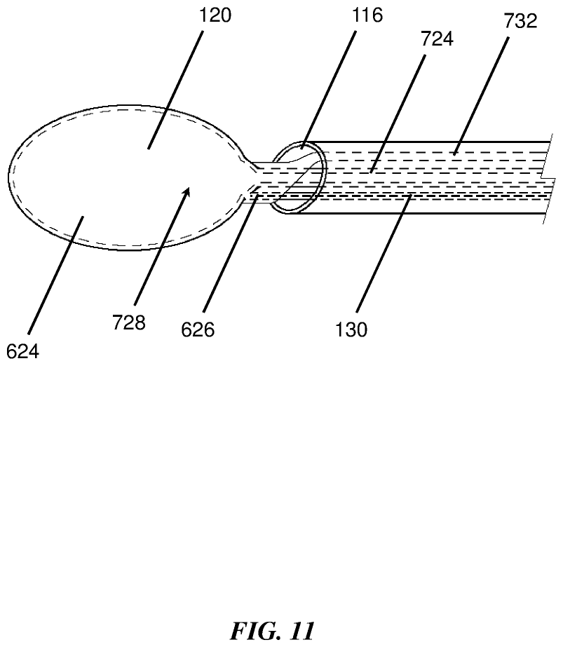

After deployment of the probe tip and expandable balloon 624, the fluid pump 716 may be used to expand the balloon 624. Assembly or preparation of the probe 110 may include connecting the fluid pump 716 to the probe 110 and filling the fluid system with a desired fluid. If saline, for example, is used, the fluid carrying components may be filled with saline and air may be removed from the system. During surgery, the fluid pump 716 is then operable to fill the expandable balloon 624 with fluid as is shown in FIG. 11. By way of example, an operator may use a fluid pump 716 such as a syringe 720 to pump fluid into the interior cavity 728 of the expandable balloon 624 via the tubing 724. The size of the expanded balloon 624 will dictate the amount of fluid used. For the fluid, various fluids have different advantages. Air has a higher compressibility but a lower thermal mass; allowing for quicker operation of the probe and a smaller heat affected tissue volume. Saline has low compressibility so the volume of the expanded balloon is more precisely controlled, but has a higher thermal mass (i.e. a higher specific heat capacity) so heating may be somewhat slower due to the heat absorbed by the saline. This may allow for a slower heating operation with the probe and a larger heat affected tissue volume.

After placement of the probe cannula 116, deployment of the probe tip 120 and expandable balloon 624, and expansion of the expandable balloon, the probe tip 120 may then be energized by delivering high frequency electricity to the probe tip electrode 736 to thereby treat the target tissue. In many instances, the tissue is thermally treated by the high frequency electricity. The treatment may scar, ablate, or cauterize the target tissue thermally. The treatment may cause death of the target issue due to heat applied to the target tissue. After the treatment is complete, the fluid pump 716 is used to withdraw the fluid from the expandable balloon 624; allowing the expandable balloon 624 to collapse and return to the state shown in FIG. 10. The lock 708 is then unlocked and the retractor 700 is used to move the retraction rod 732 and retract the probe tip 120 and expandable balloon 624 into the cannula 116. The cannula 116 may then be removed from the patient body.

In discussing the following probe tip configurations, primary attention is given to the overall shape of the probe tip 120 and the shape and location of electrode wires 736 and the corresponding active treatment zone 750. Attention is also given to the use of the probes 110 and how an active treatment zone which occupies different parts of the probe tip 120 may allow a medical practitioner to treat different target tissues 164. The various active treatment zone configurations, such as on the distal end, proximal end, the side of the probe tip 120, or the whole of the balloon or mesh basket expandable probe tip 120, give a medical practitioner the ability to more precisely treat a target tissue 164 while shielding adjacent tissue from collateral treatment. In addition to the configurations shown, each of the probe tips may be formed with different electrode materials. For example, a magnetic material may be used to form the electrode 736. In another example, a wire such as stainless wire may be used to form the electrode. In another example, a metal such as Nitinol may be used to incorporate a shape memory into the electrode wire 736.

Additionally, the probes 110 may include probe tips 120 which are configured to operate in monopolar or bipolar mode. A monopolar probe tip may have an electrode wire or multiple electrode wires which are electrically connected and which are connected to the RF power source 140. The electrode wires 736 may be connected to the power source 140 with a shielded cable 130 such that wires 130 and electrical leads 626 outside of the active treatment region 750 do not emit significant amounts of RF energy. A bipolar probe tip may separate the electrode wire or wires 736 in the active treatment region 750 into two or more electrically isolated sections which are each separately connected to the RF power source 140. One section of the electrode wire 736 provides a signal source and the other section of the electrode wire 736 provides a ground or signal return. Each of these sections of the electrode wire 736 may individually connected to an insulated or shielded electrical lead 626 and insulated or shielded connector cable 130. In another example, the portions of the wires which are not desired to be part of the active treatment region may be made from an inert material (e.g. a non-conductive or non-RF emitting material) which forms a structural part of the mesh basket by does not form part of the treatment region 750. Such a non-emitting material may be selected from the other materials described herein with respect to other embodiments. If multiple signal or ground or sections of electrode wire 736 are used in a probe tip configuration, the signal section(s) of the electrode wire 736 may be connected to a common electrical connection cable 130 and the ground section(s) of the electrode wire 736 may be connected to a common electrical connection cable 130. The signal and ground electrical cables 130 may be contained within a single cable jacket and may connect to the RF power source 140 at a single connector for ease of use.

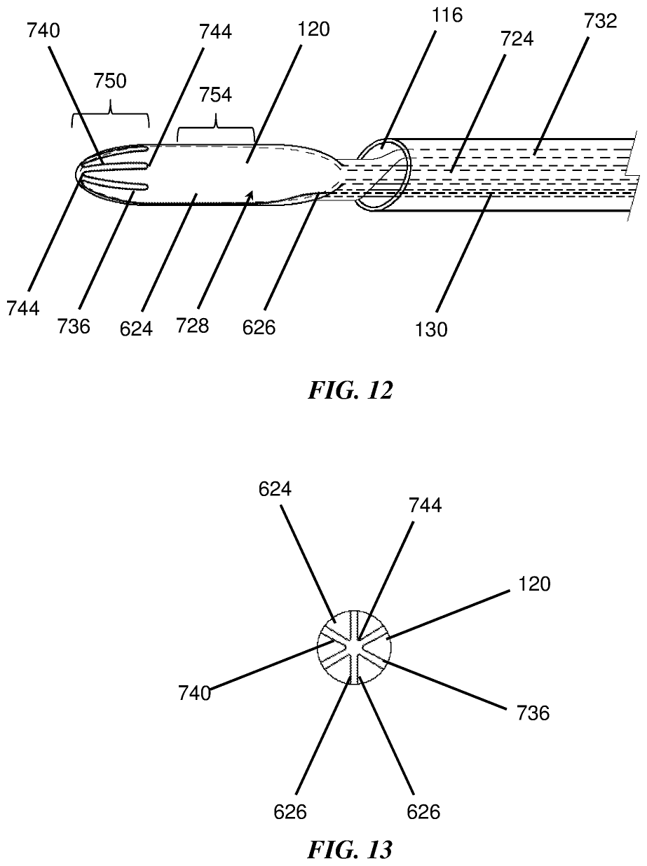

Many of the examples of probe tips 120 in FIGS. 12 through 30 show an active treatment region 750 which includes a single electrode wire 736 or which shows multiple electrode wires 736 connected together at an electrical cable 130 for operation in monopolar mode. In creating a bipolar mode probe tip 120, probes such as are shown in FIGS. 12 through 26 by separating the electrode wire 736 into isolated signal and ground electrodes by dividing the electrode wire 736 into left and right halves along a long axis of the probe tip 120. Each portion of the electrode wire 736 may be connected to an isolated electrical lead 626 and connection cable 130. Alternatively, the electrode wire 736 may be similarly divided into three isolated sections; such as a center ground section and two outer signal sections. For these figures, it is often desirable to define these sections of the electrode wire 736 along angular sections of the end of the probe tip 120 or longitudinal sections along the side of the probe tip 120.

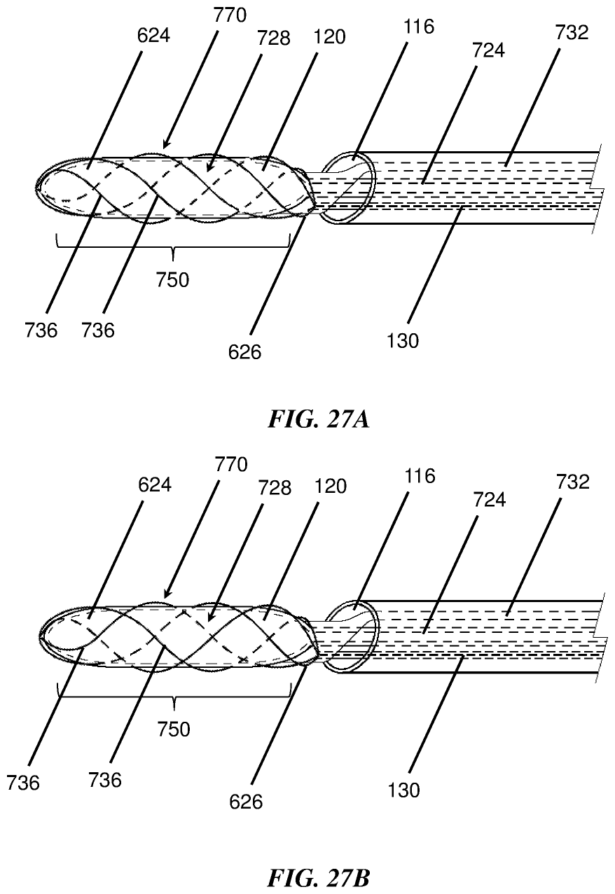

Probe tips 120 with mesh baskets such as are shown in FIGS. 27 through 30 may be separated into two or four or six isolated sections of electrode wire 736 for bipolar use by separating each loop or helix of wire into a separate signal or ground section of the electrode 736. Thus, the basket shown in FIGS. 27 through 29 may have two loops of helix wire that result in four wires spiraling around the probe tip 120. These may form one signal electrode and one ground electrode with wires that alternate circumferentially around a location along the length of the probe tip every 90 degrees (S-G-S-G). The looped configuration shown in FIG. 30 may have one or more loops which form signal electrodes and one or more loops which form ground electrodes. As another example, the expandable electrode tips 120 shown in FIGS. 12 through 30 may alternately be configured with signal electrodes 736 along the outer periphery of the balloon 624 and a ground electrode disposed inside of the balloon, such as along the central axis of the balloon 624.

Active and inactive regions may be formed on the probe tip 120 in order to create a desired size and shape of an active treatment region 750. This may be accomplished by limiting the location of electrodes 736, through the use of insulation or shielding, etc. In some examples, it may be desirable to create length of wire or a structural element that combines a length of an active material (e.g. a metal or material which will emit high frequency energy when energized by the power source 140) and a length of an inactive material which will not emit high frequency energy when the probe tip 120 is energized by the power source 120. Such a configuration may create an intact structural feature (such as a mesh basket) with an active region on only part of the probe tip 120.

FIGS. 12 and 13 show an embodiment of a probe tip 120 with an expandable balloon 624. FIG. 12 shows a side view of the probe tip 120 and FIG. 13 shows a front (distal end) view of the probe tip 120. The expandable balloon 624 is typically formed from a high temperature elastomer such as high temperature silicone. In the example shown, the expandable balloon 624 has a relatively thin wall thickness and will stretch to expand the balloon 624. The thickness of various portions of the walls of the expandable balloon 624 may vary to allow certain portions of the balloon to expand more than other portions of the balloon. For example, the expandable balloon 624 shown in FIG. 12 may have thinner sidewalls in the middle portion of the balloon and may transition to thicker sidewalls in the proximal and distal portion of the balloon. This may encourage the expandable balloon 624 to stretch more in its middle portion during expansion of the balloon 624 and form a flatter (front to back) and more disc-shaped balloon for treatment.

The expandable balloon 624 is connected to a fluid pump 716 via a fluid tubing 724 and is connected to a retractor 700 via a retraction rod 732. One or more electrical leads 626 are used to connect electrode wires 736 to the control unit 140 and energy source. The electrode wires 736 receive energy from the control unit 140 and transmit energy into the target tissue 164 for treatment. The electrode wires 736 are attached to the expandable balloon 624. As shown, the electrode wires 736 are formed in a sunburst or zig-zag pattern formed with relatively straight sections 740 and relatively narrow bends 744. The zig-zag pattern of the electrode wires 736 is formed on the front third of the expandable balloon 624, and may more generally be formed in approximately the front fourth, the front third, or the front half of the expandable balloon 624. This front section of the expandable balloon 624 forms an active treatment region 750 of the expandable balloon 624. The relatively straight sections 740 of the electrode wires 736 are oriented longitudinally along the expandable balloon 624. The narrow bends 744 are positioned both near the distal end of the expandable balloon 624 and near the proximal boundary of the active treatment area 750 of the expandable balloon 624 and connect the straight sections 740 together.

In one example, the electrode wires 736 may be disposed within the walls of the balloon 624, such as by forming the electrode wires 736 and then over-molding the expandable balloon 624 to encase the electrode wires 736 within the balloon. In another example, the electrode wires 736 may be adhered to the surface of the expandable balloon 624. In another example, the electrode wires 736 may be mechanically attached to the outside of the expandable balloon 624. The electrode wires 736 may be threaded through the surface of the expandable balloon 624 to attach the wires to the expandable balloon.

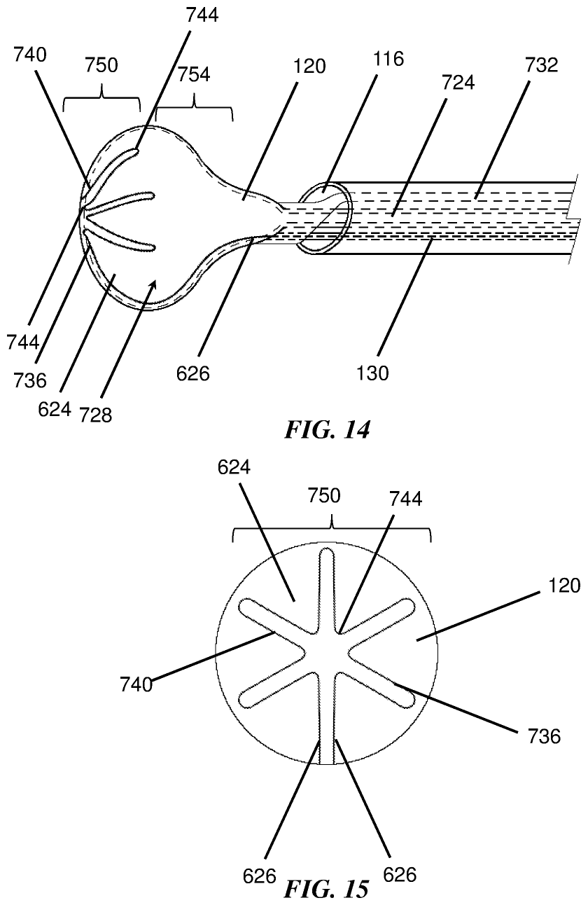

FIGS. 14 and 15 show side and end views of the probe tip 120 of FIGS. 12 and 13 with the expandable balloon 624 in an expanded state. The expandable balloon 624 is expanded during utilization of the probe tip 120 by the fluid pump 716 as discussed above. The design of the probe tip 120 promotes the integrity and efficacy of the electrode wires 736 during deployment, expansion, and use of the balloon 624. The electrode wires are oriented such that elongated lengths 740 of the electrode wire are oriented longitudinally along the balloon and such that minimal length of the electrode wire is positioned circumferentially around the balloon 624. This promotes radial expansion of the balloon. The wall thickness of the expandable balloon 624 and variation in wall thickness may also be configured to promote a desired mode of expansion of the balloon 624. As discussed, thinner walls in a middle section 754 of the balloon with thicker walls in a distal section of the balloon promotes radial expansion of the middle section 754 and a flattening of the distal section. This may minimize stretching along the length of the generally straight sections 740 of the electrode wire 736.

The electrode wire 736 shown in FIGS. 12 through 15 may be placed along the proximal end of the balloon 624 in a similar flower or star shaped configuration. Such an electrode 736 would provide an active treatment region 750 on the proximal end of the balloon 624 and allow a medical practitioner to treat a target tissue 164 adjacent the entry point of the probe cannula 116 into a structure. Such a probe tip 120 may be useful in treating a nerve or tumor in a bone where surgical access to the bone dictates entry on a particular side of the bone. A probe tip 120 may be chosen which provides an active region 750 along the distal end, side, or proximal end to align the active treatment region 750 with the target tissue 164.

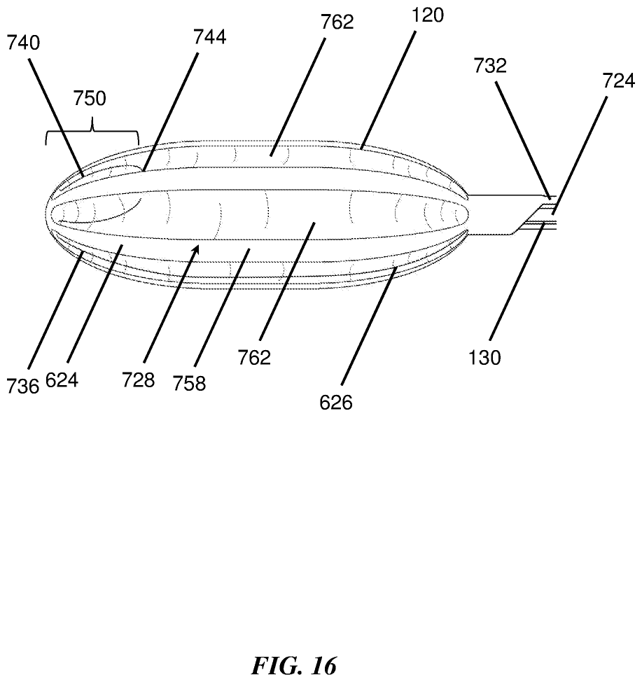

As shown in FIG. 16, the expandable balloon 624 may also include ridges 758 formed lengthwise along the expandable balloon 624. The ridges 758 control expansion of the balloon 624; causing the balloon material in thinner grooves 762 between the ridges 758 to stretch circumferentially while the thicker ridges 758 inhibit longitudinal stretching of the balloon. The electrode wire 736 may pass through the ridges 758 so that the elongate and relatively straight lengths 740 of the electrode wire 736 are disposed between the ridges 758 and the small bends 744 are formed at the ridges 758 where the electrode wire passes through the ridges 758. This configuration both stabilizes and protects the electrode wire 736 and also controls the shape of the balloon 624 during expansion.

The expanded balloon 624 provides a probe tip 120 with an active treatment region 750 which is located on the front of the balloon 624. This allows the balloon 624 to be pressed against a target tissue 164 to treat tissue in front of the balloon 624 while shielding tissue behind the balloon from treatment. This allows the medical practitioner to focus the treatment on a target tissue while minimizing effects on surrounding tissue. The active treatment region 750 is approximately the front third of the distal probe tip 120. It is typically desirable to have an active treatment region which occupies between about one fifth of the front of the probe tip 120 and about one half of the front of the probe tip 120. The active treatment region 750 often occupies approximately the front one fourth of the probe tip 120 or the front one third of the probe tip 120. The proximal portion of the probe tip 120 located proximally of the active treatment region 750 is inactive and does not comprise an active treatment region. The wires 626 which are outside of the active treatment region 750 and connected to the electrode wires 736 may be insulated with a ground shield or a thermal insulation to inhibit treatment of tissue adjacent these wires.

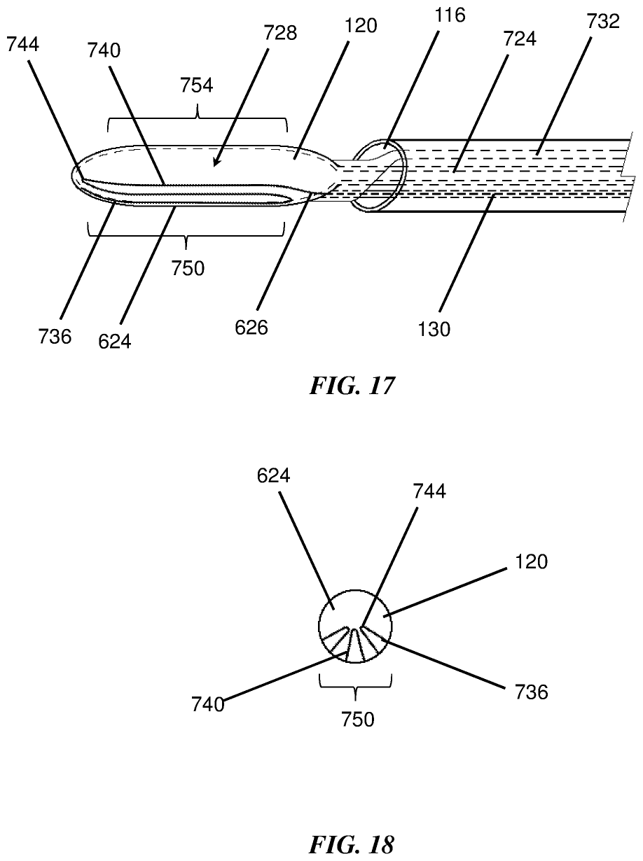

FIGS. 17 and 18 show another embodiment of a probe tip 120 with an expandable balloon 624. FIG. 17 shows a side view of the probe tip 120 and FIG. 18 shows a front view of the probe tip 120. The expandable balloon 624 is typically formed from a high temperature elastomer such as high temperature silicone. In the example shown, the expandable balloon 624 has a relatively thin wall thickness and will stretch to expand the balloon 624. The thickness of various portions of the walls of the expandable balloon 624 may vary to allow certain portions of the balloon to expand more than other portions of the balloon. For example, the expandable balloon 624 may have thinner sidewalls along the length of the balloon and may transition to thicker sidewalls in the proximal and distal end portions of the balloon. This may encourage the expandable balloon 624 to stretch more evenly throughout its middle portion during expansion of the balloon 624 and retain rounded proximal and distal ends.

The expandable balloon 624 is connected to a fluid pump 716 via a fluid tubing 724 and is connected to a retractor 700 via a retraction rod 732. One or more electrical leads 626 are used to connect electrode wires 736 to the control unit 140 and energy source. The electrode wires 736 receive energy from the control unit 140 and transmit energy into the target tissue 164 for treatment. The electrode wires 736 are attached to the expandable balloon 624. As shown, the electrode wires 736 are formed in a zig-zag pattern formed with relatively straight sections 740 and relatively narrow bends 744. The zig-zag pattern of the electrode wires 736 is formed along most of or a desired portion of the length of the balloon around approximately one third of the circumference of the expandable balloon 624. Accordingly, the expandable balloon 624 provides an active treatment region 750 on generally one side of the expandable balloon 624. This allows the probe tip 120 to treat tissue disposed along a side of the expandable balloon 624 without treating tissue on an opposite side of the expandable balloon 624. The length of the active region 750 may be varied according to application. The probe tip 120 may be formed with an active region 750 which is generally on one side of the expandable balloon 624 and which extends along the front half, the middle half, or most of the length of the expandable balloon 624, for example. The relatively straight sections 740 of the electrode wires 736 are oriented longitudinally along the expandable balloon 624. The narrow bends 744 are positioned near the proximal and distal boundaries of the active region 750 and are positioned near the distal end and proximal end of the expandable balloon 624.

In one example, the electrode wires 736 may be disposed within the walls of the balloon 624, such as by forming the electrode wires 736 and then over-molding the expandable balloon 624 to encase the electrode wires 736 within the balloon. In another example, the electrode wires 736 may be adhered to the surface of the expandable balloon 624. In another example, the electrode wires 736 may be mechanically attached to the outside of the expandable balloon 624. The electrode wires 736 may be threaded through the surface of the expandable balloon 624 to attach the wires to the expandable balloon.

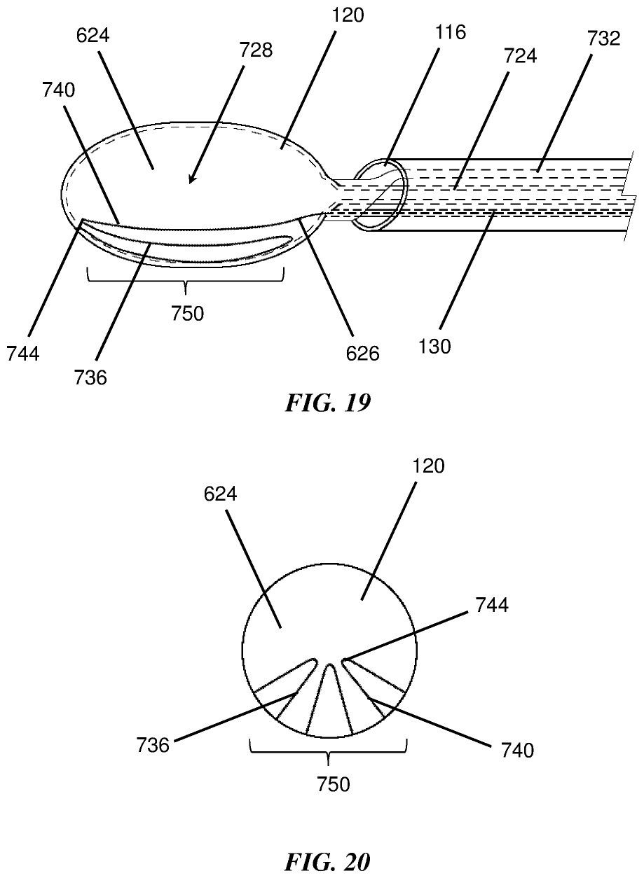

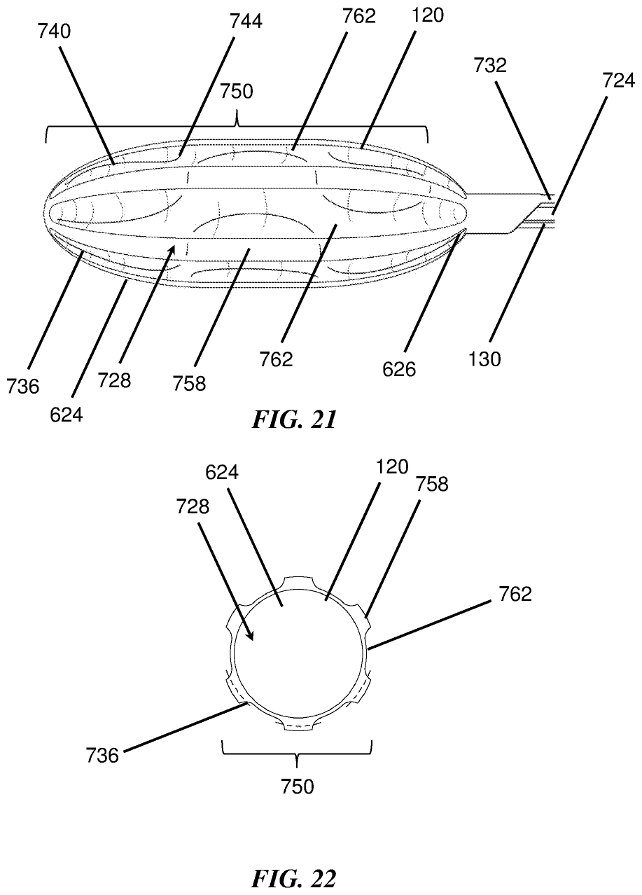

FIGS. 19 and 20 show side and end views of the probe tip 120 of FIGS. 17 and 18 with the expandable balloon 624 in an expanded state. The expandable balloon 624 is expanded during utilization of the probe tip 120 by the fluid pump 716 as discussed above. The design of the probe tip 120 promotes the integrity and efficacy of the electrode wires 736 during deployment, expansion, and use of the balloon 624. The electrode wires are oriented such that elongated lengths 740 of the electrode wire are oriented longitudinally along the balloon and such that minimal length of the electrode wire is positioned circumferentially around the balloon 624. This promotes radial expansion of the balloon. The wall thickness of the expandable balloon 624 and variation in wall thickness may also be configured to promote a desired mode of expansion of the balloon 624. As discussed, thinner walls in a middle section 754 of the balloon with thicker walls in a distal section of the balloon promotes radial expansion of the middle section 754 and a flattening of the distal section. This may minimize stretching along the length of the generally straight sections 740 of the electrode wire 736. As shown in FIGS. 21 and 22, the expandable balloon 624 may also include ridges 758 formed lengthwise along the expandable balloon 624. FIG. 22 shows a cross section of the expandable balloon 624 of FIG. 22. The ridges 758 control expansion of the balloon 624; causing the thinner balloon material 762 between the ridges 758 to stretch circumferentially while the thicker ridges 758 inhibit longitudinal stretching of the balloon. The electrode wire 736 may pass through the ridges 758 so that the elongate and relatively straight lengths 740 of the electrode wire 736 are disposed between the ridges 758 and the small bends 744 are formed at the ridges 758 where the electrode wire passes through the ridges 758. This configuration both stabilizes and protects the electrode wire 736 and also controls the shape of the balloon 624 during expansion.

The expanded balloon 624 provides a probe tip 120 with an active treatment region 750 which is located along a desired length of a side of the balloon 624. This allows the side of the balloon 624 to be pressed against a target tissue 164 to treat tissue along the side of the balloon 624 while shielding tissue on an opposite side of the balloon from treatment. This allows the medical practitioner to focus the treatment on a target tissue while minimizing effects on surrounding tissue. The active treatment region 750 occupies a side of the probe tip 120 and occupies approximately one third of the circumference of the middle of the probe tip 120. For this probe tip configuration, the active treatment region typically occupies between approximately one fourth of the circumference of the probe tip and approximately one half of the circumference of the probe tip. The side of the probe tip 120 opposite the active treatment region 750 is not an active treatment region. If necessary, electrode wires 736 or electrical leads 626 connecting to electrode wires outside of the active treatment region may be shielded or insulated.

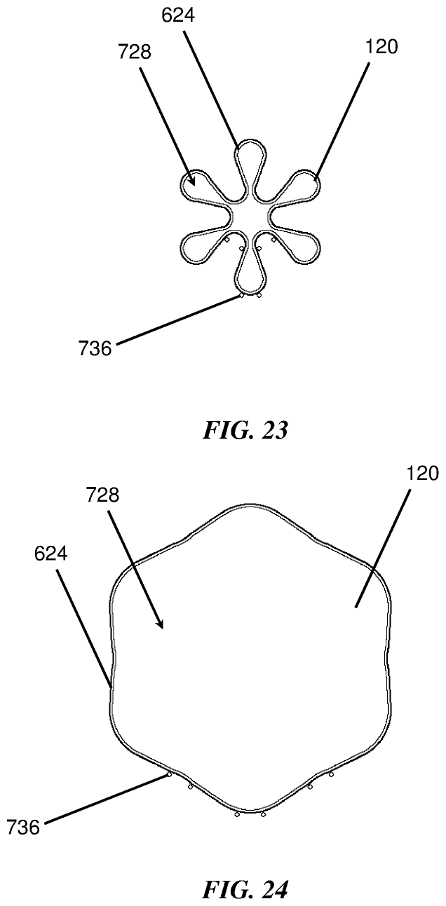

FIGS. 23 and 24 show alternate cross-sectional views of a balloon 624 such as shown in FIG. 12 or 17. FIG. 23 shows the balloon 624 in a collapsed state and FIG. 24 shows the balloon 624 in an expanded state. The expandable balloon 624 may be formed so that the balloon has a folded or corrugated circumferential configuration; having inward and outward folds. Introduction of fluid into the balloon 624 will expand the balloon by flexing its walls into the position shown in FIG. 24. This expandable balloon 624 may expand with less pressure than other expandable balloons as the balloon need not stretch to the degree required for other balloon configurations. The electrode wires 736 may be configured as shown in FIG. 12 or 17, for example, to provide active portions along the distal end, proximal end, or side of the expandable balloon 624.

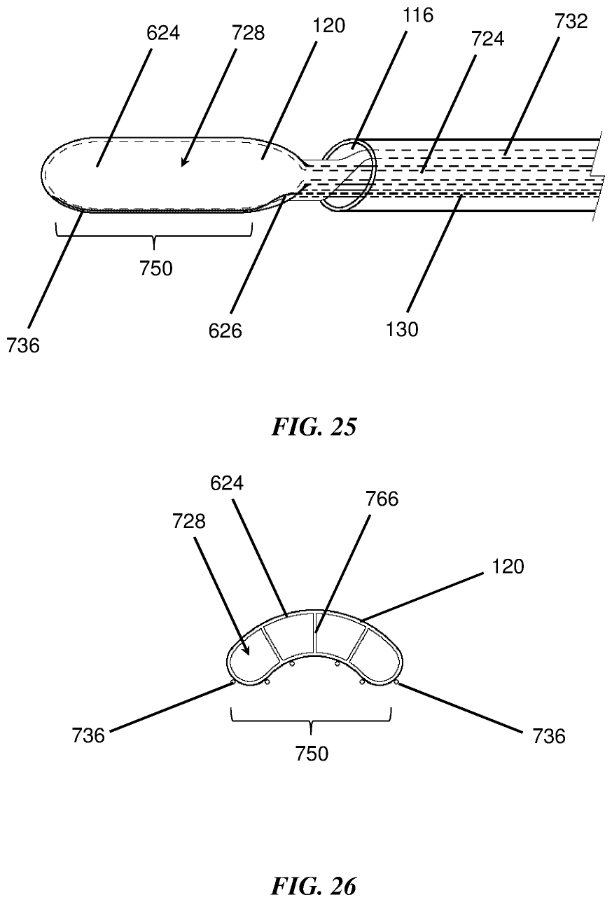

FIGS. 25 and 26 show another embodiment of a probe tip 120 with an expandable balloon 624. FIG. 25 shows a side view of the probe tip 120 and FIG. 26 shows a front view of the probe tip 120. The expandable balloon 624 is typically formed from a high temperature elastomer such as high temperature silicone. In the example shown, the expandable balloon 624 is formed in a crescent shape and includes interior walls 766 to allow the expandable balloon 624 to form a crescent shaped balloon as viewed from its distal end. The balloon shape allows a medical practitioner to treat nerves or tissue along the outer surface of a bone, for example.

Electrical leads 626 are connect electrode wires 736 which are attached to the expandable balloon 624 on the distal and inside surfaces of the crescent shape. As shown, the electrode wires 736 are formed in a zig-zag pattern along a distal portion of the interior of the crescent shape which begins adjacent the front of the balloon 624 and extends proximally within an active treatment region 750 of the probe tip 120. The electrode wires 736 are formed with relatively straight sections 740 and relatively narrow bends 744.

The expandable balloon 624 is expanded during utilization of the probe tip 120 by the fluid pump 716 as discussed above. The design of the probe tip 120 promotes the integrity and efficacy of the electrode wires 736 during deployment, expansion, and use of the balloon 624. The electrode wires are oriented such that elongated lengths 740 of the electrode wire are oriented longitudinally along the concave channel formed along the balloon 624 where they may be utilized to treat the exterior of a convex surface such as a bone. The expandable balloon 624 may also include ridges formed lengthwise along the expandable balloon 624 and the electrode wire 736 may pass through the ridges 758 so that the elongate and relatively straight lengths 740 of the electrode wire 736 are disposed between the ridges 758 and the small bends 744 are formed at the ridges 758 where the electrode wire passes through the ridges 758.

In certain embodiments, an RFA probe 120 may be formed from a mesh web made of one or more metals (e.g. a “metal mesh web”). The metal mesh web is flexible and can conform to the shape of tissue under treatment. The metal mesh web may be configured in a wide variety of shapes and sizes depending on the clinical application for which the metal mesh web is manufactured and/or used. As one example, the metal mesh web may be shaped like a basket. The metal mesh web may include regions that are active, and regions that are inert. The active regions deliver electromagnetic (such as thermal) radiation to the tissue under treatment, while the inert regions provide little or no heating (or other forms of radiation) to the tissue under treatment. The presence of the inert material may reduce unintended damage to tissue adjacent to the tissue under treatment. In embodiments, the active regions of the metal mesh web may be configured to provide or otherwise operate as an RFA probe in either a monopolar or bipolar mode.

In certain embodiments, the metal mesh web may have regions made from different types of materials. For example, a metal mesh web may be fabricated from one or more of ferromagnetic, ferrite, or nonmagnetic materials. Further, different regions of the metal mesh web may be energized by one or more different electrical circuits. In some embodiments, a metal mesh web may be positioned at a distal end of an RFA probe. In embodiments, a metal mesh web may be integrated with an expander that opens, or otherwise expands the metal mesh web in order to increase the area and/or volume of the metal mesh web probe. In embodiments, the metal mesh web and expander may be positioned at a distal end of an RFA probe. In embodiments, a metal mesh web and expander may be positioned on a retractable tip that may be deployed from within a hollow probe body, and then retracted following treatment.

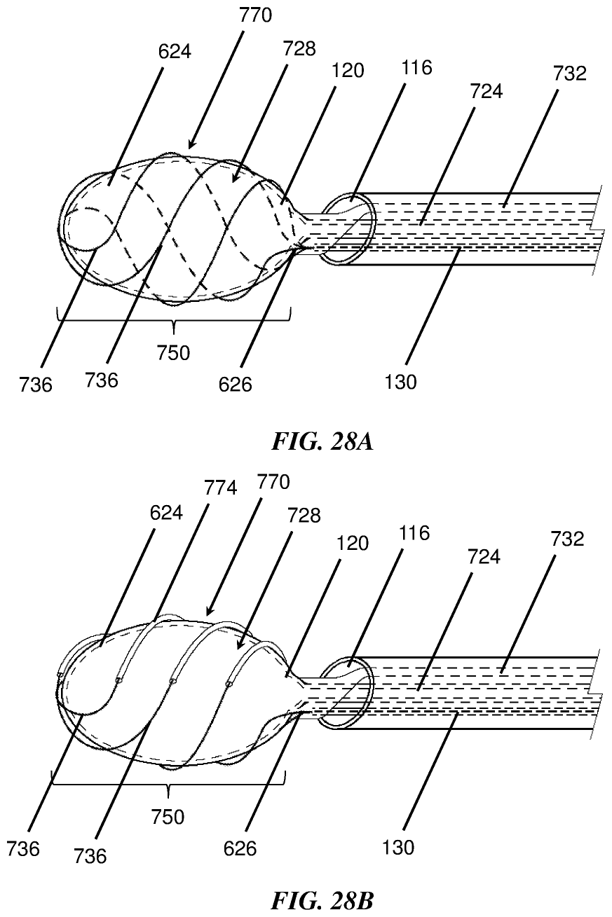

FIGS. 27A, 27B, 28A, and 28B show an embodiment of a probe tip 120 with a mesh basket 770. The probe tip 120 may also include an expandable balloon 624. FIGS. 27A and 27B show side views of the probe tip 120 with the probe tip 120 in a collapsed state and FIG. 28A and 28B show side views of the probe tip 120 in an expanded state. The probe tip 120 includes one or more electrode wires 736 which are formed into a cylindrical or tapered cylindrical shape. The example includes two loops of electrode wire 736. Each electrode wire 736 is twisted into a terminated helix shape such that the wire spirals towards the distal end of the probe tip 120, curves across the distal end of the probe tip, and spirals back to the proximal end of the probe tip. One electrode wire 736 may be formed into a right hand helix and the other electrode wire 736 may be formed into a left hand helix and the two electrode wires may be woven together as they cross each other (FIG. 27B). Alternately, both electrode wires may be formed into a right or a left hand helix such that they run parallel to each other and do not cross each other as they spiral towards and away from the distal end (FIG. 27A). Such a probe tip may be formed of a single helix electrode wire or two or more helix electrode wires which spiral the same direction or in opposite directions and are woven.

At the proximal end, each end of the electrode wires 736 may be connected together electrically. For a probe tip operating in a monopolar RF mode, the electrode wires 736 may be joined together electrically, such as adjacent the base of an expandable balloon 624 or adjacent the proximal end of the probe tip 120. The electrode wire(s) 736 and the ground pad 190 may be connected to the oscillating power supply 140 such that electricity flows through the connector cables 130, probe tip 120, patient tissue under treatment 164, and the ground pad 190.

Each of the probe tips 120 with an expandable balloon 624 may be operated in a monopolar RF mode in this manner with one or more electrode wires 736 in an active region 750 which are electrically connected to each other and connected to the oscillating energy source 140 via a connector cable 130. For each of the probe tips 120 with an expandable balloon 624 or which forms an expandable basket, expandable mesh, or expandable volume, the electrode wires 736 may be formed from a stainless steel wire or from other biologically compatible materials. In some examples, Nitinol may be used for the electrode wires. Nitinol wire may be heat treated to set the wire into a desired shape and the wire retains shape memory to return to that shape.

The electrode wire 736 may be connected to one or more electrical leads 626 (typically one for monopolar use) which are in turn connected to the connector cable 130 and power source 140. The probe tip 120 is connected to a retraction rod 732 which is connected to the retractor 700; allowing the probe tip to be deployed from a cannula 116 and retracted into the cannula 116.

The helically spiraled or helically woven electrode wires 736 form a mesh basket which generally encloses an interior volume. An advantage of a helically spiraled or loosely helically woven mesh basket is that the basket will change shape in response to applied force. For example, pressing the distal end of the mesh basket will compress the mesh baskets length and expand its diameter. FIG. 28A shows the mesh basket probe tip 120 of FIG. 27 in a diametrically expanded state. The electrode wires 736 are expanded radially and the area covered by the distal end of the mesh basket is increased. In this manner, the mesh basket probe tip 120 will conform to tissue as it is pressed against the tissue and forms a desirable delivery method for RF energy.

The mesh basket probe tip 120 may also include an expandable balloon 624. The expandable balloon 624 is connected to a fluid pump 716 via a fluid tubing 724 and may also be connected to the retraction rod 732. The expandable balloon 624 is typically formed from a high temperature elastomer such as high temperature silicone. In the example shown, the expandable balloon 624 has a relatively thin wall thickness and will stretch to expand the balloon 624. The thickness of various portions of the walls of the expandable balloon 624 may vary to allow certain portions of the balloon to expand more than other portions of the balloon. For example, the expandable balloon 624 may have thinner sidewalls in the middle portion of the balloon and may transition to thicker sidewalls in the proximal and distal portion of the balloon. This may encourage the expandable balloon 624 to stretch more in its middle portion during expansion of the balloon 624 and form a flatter (front to back) and more spherical or disc-shaped balloon for treatment. Expansion of the expandable balloon 624 will simultaneously expand the mesh basket electrode wires 736. The probe tip 120 may be deployed from a cannula 116 at a desired location within a body, expanded to treat tissue, collapsed, retracted into the cannula 116, and removed from the body.

The mesh basket probe tip 120 is advantageous as it provides a geometrically stable configuration of electrode wires. The electrode wires 736 are stabilized by their helical twist. Inclusion of an expandable balloon 624 further stabilizes the helical electrode wires 736 around the balloon. The electrical wires 736 need not be attached to the balloon 624 as the balloon is held captive within the electrode wires so construction of the probe tip is simplified.

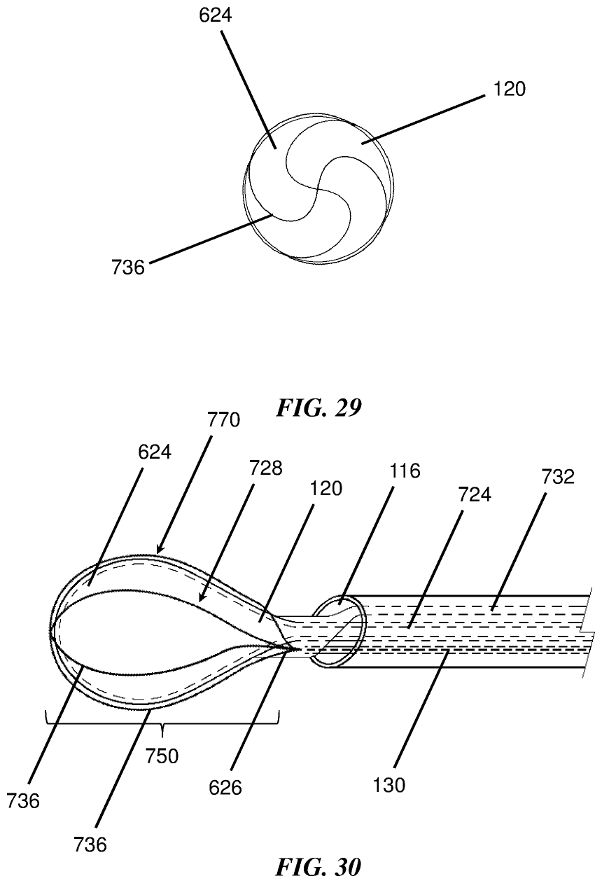

FIG. 29 shows an end view of the probe tip 120 of FIGS. 27A, 27B, 28A, and 28B with the expandable balloon 624 in an expanded state. The expandable balloon 624 is expanded during utilization of the probe tip 120 by the fluid pump 716 as discussed above. If desired, the expandable balloon 624 may also include ridges 758 formed along the expandable balloon 624. The ridges 758 control expansion of the balloon 624; causing the thinner balloon material between the ridges 758 to stretch while the thicker ridges 758 inhibit stretching of the balloon along the ridges.

The expanded balloon 624 or mesh basket provides a probe tip 120 with an active treatment region 750 which is located on the front and sides of the balloon 624. This allows the balloon 624 to be pressed against a target tissue 164 to treat tissue in front of or along the sides of the balloon 624. If desired, a probe tip 120 can be made which limits the active treatment region 750 to a particular area of the probe tip 120 by insulating the electrode wires. FIG. 28B shows how insulation 774 may be used to limit the active treatment region 750 of the probe tip 120 to a desired location on the probe tip. The insulation 774 may be a thermal insulation such as close fitting tubular high temperature silicone which may be formed or placed around the electrode wires 736 to insulate a portion of the electrode wires 736. The insulation 774 may also be an electrical insulation such as a ground shielding which limits the transmission of RF energy from a portion of the electrode wire 736. Such an insulation 774 may include a cylindrically tubular insulation material such as high temperature silicone or inert polymer material surrounding the electrode wire and an electrically conductive (e.g. metal) ground surrounding the tubular insulation material to inhibit the propagation of RF energy from the insulated section of electrode wire. The insulation is shown along one of the sides of the probe tip 120 in order to create an active region 750 along the opposite side of the probe tip 120. Tissue adjacent the insulated side of the probe tip 120 is shielded from the treatment effects of the probe tip 120. The insulation 774 may alternatively be placed adjacent the proximal end of the probe tip 120 to shield tissue adjacent the proximal end of the probe tip 120 from treatment, creating an active zone 750 around the sides of the probe tip 120. The insulation 774 may alternatively be placed around the electrode wires 736 along the sides of the probe tip 120 so that tissue adjacent the sides of the probe tip 120 are shielded from treatment and the active treatment region 750 is focused at the distal end of the probe tip 120. Alternatively, the distal end of the probe tip 120 and one side of the probe tip 120 may include insulation 774 so that the active region 750 is focused along one of the sides of the probe tip 120.

FIG. 30 shows a side view of another embodiment of a probe tip 120 with a mesh basket 770 in an expanded state. In a non-expanded state, the probe tip 120 is somewhat longer and smaller in diameter. The probe tip 120 may also include an expandable balloon 624 disposed inside of the mesh basket of electrode wires 736. The probe tip 120 includes one or more electrode wires 736 which are formed into loops which extend forwards to the distal end of the probe tip 120. The probe tip 120 may typically include two, three, or four electrode wires 736. Each electrode wire 736 extends towards the distal end of the probe tip 120, curves across the distal end of the probe tip, and extends back to the proximal end of the probe tip.

At the proximal end of the probe tip 120, each end of the individual electrode wires 736 may be connected together mechanically and electrically. For a probe tip operating in a monopolar RF mode, the electrode wires 736 may be joined together electrically, such as adjacent the base of the expandable balloon 624 adjacent the proximal end of the probe tip 120. The electrode wire(s) 736 and the ground pad 190 may be connected to the oscillating power supply 140 such that electricity flows through the connector cables 130, probe tip 120, patient tissue under treatment 164, and the ground pad 190.

The electrode wires 736 of this and the other basket mesh probe tips may be formed from a stainless steel wire or from other biologically compatible materials. Nitinol may be used for the electrode wires 736 as Nitinol wire may be heat treated to set the wire into a desired shape and the wire retains shape memory to return to that shape. The electrode wire 736 may be connected to one or more electrical leads 626 (typically one for monopolar use) which are in turn connected to the connector cable 130 and power source 140. The probe tip 120 is connected to a retraction rod 732 which is connected to the retractor 700; allowing the probe tip to be deployed from a cannula 116 and retracted into the cannula 116.

The looped electrode wires 736 form a mesh basket which generally encloses an interior volume. The mesh basket will change shape in response to applied force. For example, pressing the distal end of the mesh basket will compress the mesh basket along its length and expand its diameter. The electrode wires 736 are expanded radially outwardly and the area covered by the distal end of the mesh basket is increased. In this manner, the mesh basket probe tip 120 will conform to tissue as it is pressed against the tissue and forms a desirable delivery method for RF energy.

The expandable balloon 624 is connected to a fluid pump 716 via a fluid tubing 724 and may also be connected to the retraction rod 732. The expandable balloon 624 is typically formed from a high temperature elastomer such as high temperature silicone. In the example shown, the expandable balloon 624 has a relatively thin wall thickness and will stretch to expand the balloon 624. The thickness of various portions of the walls of the expandable balloon 624 may vary to allow certain portions of the balloon to expand more than other portions of the balloon. For example, the expandable balloon 624 may have thinner sidewalls in the middle portion of the balloon and may transition to thicker sidewalls in the proximal and distal portion of the balloon. This may encourage the expandable balloon 624 to stretch more in its middle portion during expansion of the balloon 624 and form a flatter (front to back) and more spherical or disc-shaped balloon for treatment. Expansion of the expandable balloon 624 will simultaneously expand the mesh basket electrode wires 736. The probe tip 120 may be deployed from a cannula 116 at a desired location within a body, expanded to treat tissue, collapsed, retracted into the cannula 116, and removed from the body.

The mesh basket probe tip 120 is advantageous as it provides a geometrically stable configuration of electrode wires. The loops of electrode wires 736 are stable and inclusion of an expandable balloon 624 further stabilizes the helical electrode wires 736 around the balloon. The electrical wires 736 need not be attached to the balloon 624 as the balloon is held captive within the electrode wires so construction of the probe tip is simplified.

The expanded balloon 624 or mesh basket provides a probe tip 120 with an active treatment region 750 which is located on the front and sides of the balloon 624. This allows the balloon 624 to be pressed against a target tissue 164 to treat tissue in front of or along the sides of the balloon 624. If desired, a probe tip 120 can be made which limits the active treatment region 750 to a particular area of the probe tip 120 by insulating the electrode wires. For example, close fitting tubular high temperature silicone may be formed or placed around the electrode wires 736 adjacent the proximal end of the probe tip 120 to shield tissue adjacent the proximal end of the probe tip 120 from the treatment. This insulation may also be placed around the electrode wires 736 along the sides of the probe tip 120 so that tissue adjacent the sides of the probe tip 120 are shielded from treatment and the active treatment region 750 is focused at the distal end of the probe tip 120. Alternatively, the distal end of the probe tip 120 may include this insulation so that the active region 750 is focused along the sides of the probe tip. Further yet, this insulation may be placed around the electrode wires 736 along one side of the probe tip 120 so that the active treatment region 750 is focused along an opposite side of the probe tip 120.

The various mesh basket probe tips 120 discussed above are advantageous in that the electrode wires 736 will expand radially to a larger size when pressed against a tissue or with the assistance of an expandable balloon 624. The electrode wires 736 form a stable configuration and will retain their configuration with minimal need if any for attaching the electrode wires 736 to the balloon and to each other. The electrode wires 736 are also designed such that they will collapse to a smaller overall diameter when placed under tension. Accordingly, pulling the probe tip 120 into the cannula 116 with a retractor rod 732 will urge the probe tip 120 into an elongate configuration of reduced diameter. This encourages a clean and easy retrieval of the probe tip 120 after a treatment has been performed.

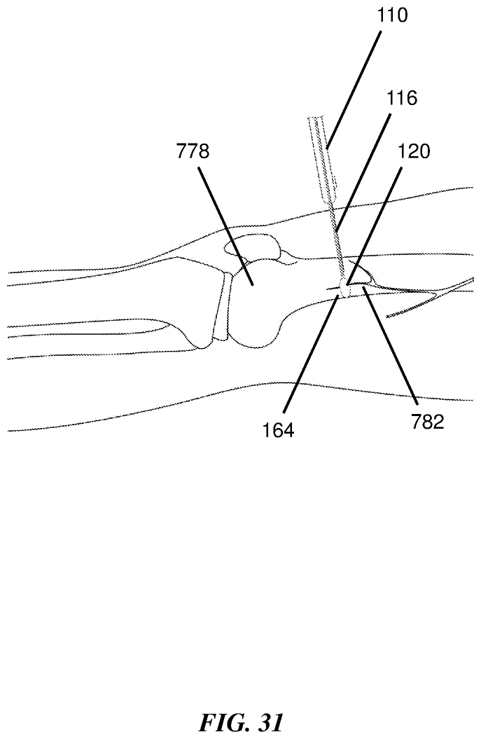

FIGS. 31 through 26 show example treatment procedures using the probes 110 and probe tips 120 described herein. The example treatment procedures particularly show applications where it is advantageous to use a probe 110 with a cannula 116 and a deployable and expandable probe tip 120. The description of each example procedure is compatible with each of the probes shown in FIGS. 12 through 30 and may particularly benefit from a particular configuration of electrode wire 736 or active treatment region 750 in order to place the treatment region at the distal end or side of the probe tip 120. In each example, the probe cannula 116 is inserted into a desired location in a body tissue, the probe tip 120 is deployed and expanded, and the target tissue is treated by emitting high frequency electrical energy from the probe tip active treatment region 750.

FIG. 31 shows an example treatment procedure using the RFA probe 110. A side view drawing of a knee is shown highlighting the femur and the genicular nerve. In some instances, it is desirable to ablate the genicular nerve 782 to provide relief from chronic pain. The genicular nerve may be found in varied locations at the femur 778. Accordingly, it may be difficult to precisely locate for treatment. A probe 110 with an elongate balloon or mesh basket probe tip 120 such as is shown in FIG. 18 or 28 may be used to ablate the genicular nerve at the femur and provide relief from chronic pain. A balloon or mesh basket probe tip 120 with an elongate active region 750 extending along one side of the probe tip 120 may be advantageously used to ablate the nerve at the femur 778. Such a probe tip 120 can deliver treatment to a longer area along the surface of the femur 778 in a single treatment application after insertion of the cannula 116, deployment of the probe tip 120, and expansion of the balloon or basket. To the contrary, treatment with a probe tip 120 which is active at a single point may require a number of ablation treatments at different locations to ensure ablation of the nerve. Thus, the elongate active region 750 provides a single treatment procedure which covers an elongate target region along the bone 778 and is less likely to miss the genicular nerve 782. Additionally, a probe tip 120 with an active region 750 on one side of the tip shields tissue on the other side of the probe tip 120 from the treatment and minimizes collateral damage to surrounding tissue.

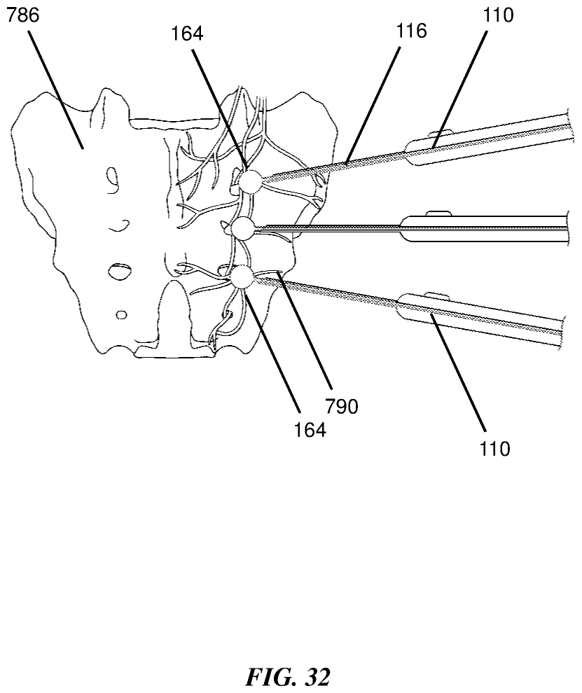

FIG. 32 shows how a probe 110 with a balloon or basket probe tip 120 may be similarly used to treat the sacral lateral branch nerve at the sacroiliac joint. The sacrum 786 and lateral branch nerve tissue 790 is shown. A probe with a balloon or basket probe tip 120 that provides an active area 750 on its tip or which provides a crescent shape with a distal active area may be used to target tissue 164 at sacral lateral branch nerves 790. This nerve tissue can also be better targeted with a balloon or basket probe tip which can cover a larger area with a single treatment and ensure ablation of the desired tissue.

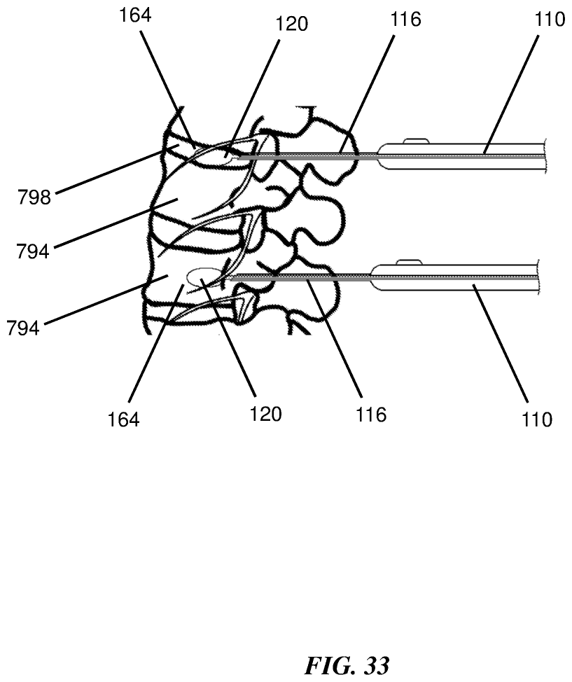

FIG. 33 shows how a probe 110 with a balloon or basket probe tip 120 may be used to perform ablation within a vertebra 794 or disc 798. For intradiscal ablation, a probe 110 with a balloon or basket tip 120 where substantially the entire balloon or basket is an active treatment region 750 may be used. The cannula 116 may be inserted into the interior of a disc 798 (typically from a posterior-lateral approach), the probe tip 120 deployed, the balloon or basket expanded to correspond to the desired treatment volume, and the probe tip 120 energized to treat the target tissue. The probe tip 120 may then be collapsed and retracted into the cannula 116 and the probe 110 withdrawn from the patient. For such an application where the cannula 116 penetrates a durable tissue, a sharpened cannula tip is typically preferred. For vertebral body ablation, a sharpened cannula 116 may be inserted into a vertebral body 794, the tip 120 deployed via the retractor, the basket or balloon expanded to the desired size, and the tip 120 energized to ablate the target tissue. Such a procedure is useful for treatment of a tumor within the interior of a vertebra 794 or another bone. In most of these applications, a tip 120 with substantially all of the basket or balloon forming an active region 750 is preferred.

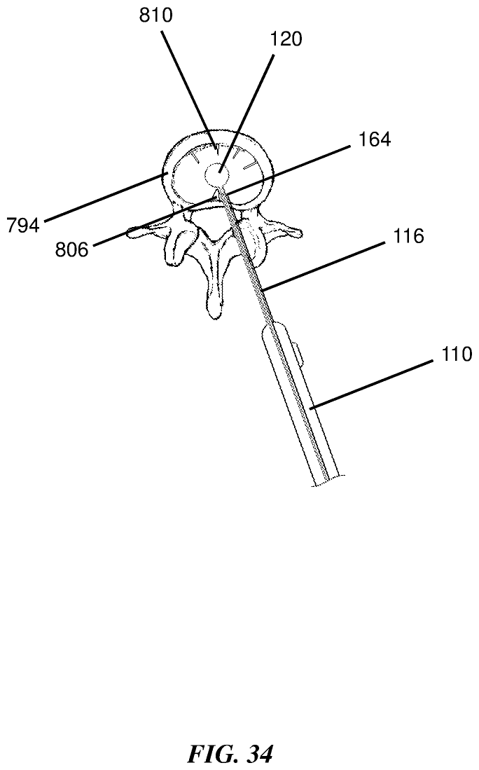

A similar procedure may be used to ablate a nerve which innervates the bone, such as a basal vertebral nerve 806 (FIG. 34) and peripheral nerves 810 which innervate the vertebral endplate. This procedure may also involve insertion of a probe cannula 116 through a posterior/lateral approach into the vertebral body 794, deployment, expansion, and energization of the probe tip 120. The balloon or basket probe tip 120 may be used with an overall active treatment area 750 which covers substantially all of the basket or balloon. Such a treatment area may ablate both the basal vertebral nerve 806 and the peripheral nerves 810. Alternatively, the probe tip 120 may include a balloon or basket with an active area on its posterior end and/or the posterior portion of its sides. Such a probe tip will focus the ablation at the posterior or posterior lateral side of the vertebra 794 and focus treatment on the basal vertebral nerve 806. In this manner, a tip 120 with a balloon or basket with an active region 750 at the posterior end of the balloon or basket may be used to ablate tissue adjacent to the entry point of the cannula into the bone or target tissue and may allow the treatment to be focused on an area which would otherwise be difficult to access.

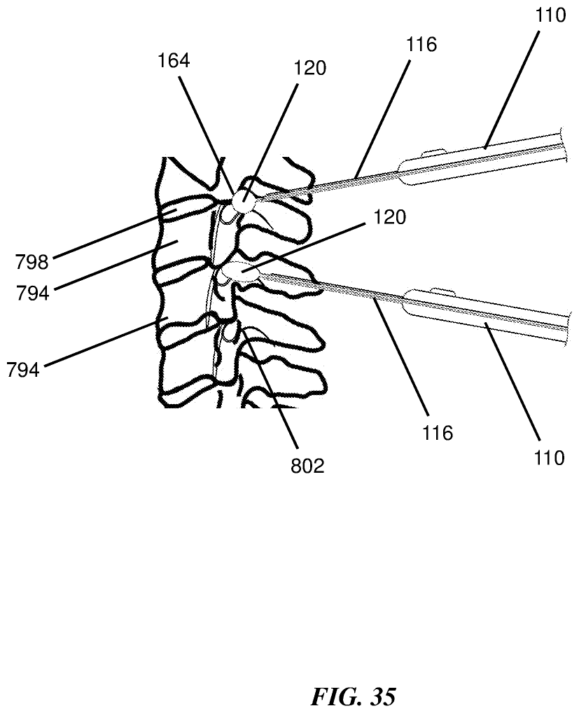

FIG. 35 illustrates how a probe 110 with an expandable balloon or basket tip 120 may be used to treat nerves at a cervical facet 802 or along the cervical facet column. In particular, the probe 110 may be used to treat medial branch nerves or the third occipital nerve along the lateral facet column. A cannula 116 with a probe tip 120 having an elongate active treatment region 750 along the side of the probe tip 120 may be inserted into a body via a posterior approach generally parallel to the surface of the facet. The probe tip 120 is deployed and expanded such that the active treatment region 750 is pressed against the surface of a target zone along the facet column. The active treatment region 750 may be used to treat facet nerves such as the medial branch nerves. This is illustrated by the lower probe 110 in FIG. 35. Alternatively, a cannula 116 with a probe tip 120 having a rounded or blunt balloon or basket with an active treatment region 750 on the distal end or distal half of the probe tip 120 may be inserted into a body via a posterior approach generally perpendicular to the surface of the bone along the facet column. The probe tip 120 is deployed and expanded such that the probe tip 120 is pressed against the surface of the facet column at the target area. The balloon or basket provides an active treatment region 750 focused on the tip of the balloon or basket. Pressing the balloon or basket onto the surface of the bone conforms the tip of the balloon or basket onto the facet surface and allows a broader area to be treated in one treatment while adequately covering the area to achieve the desired effect. This may be particularly effective in treating the third occipital nerve which lies across a convex region of bone. The expandable probe tip 120 is useful in applications such as the cervical facet column where the target tissue lies across a bone with an uneven surface shape. These bone surfaces may be convex, concave, or uneven surfaces. The probe tip 120 conforms to the surface of the bone and provides treatment to the desired area. In this manner, the balloon or basket tip 120 allows a medical practitioner to provide treatment uniformly across a broader region. This provides improved outcomes where a particular tissue such as a nerve cannot be precisely located or where a larger region of tissue must be treated. The probe tip 120 is effective in providing treatment across a region of bone, for example, where the surface of the target tissue is uneven as the probe tip 120 will conform to the surface.

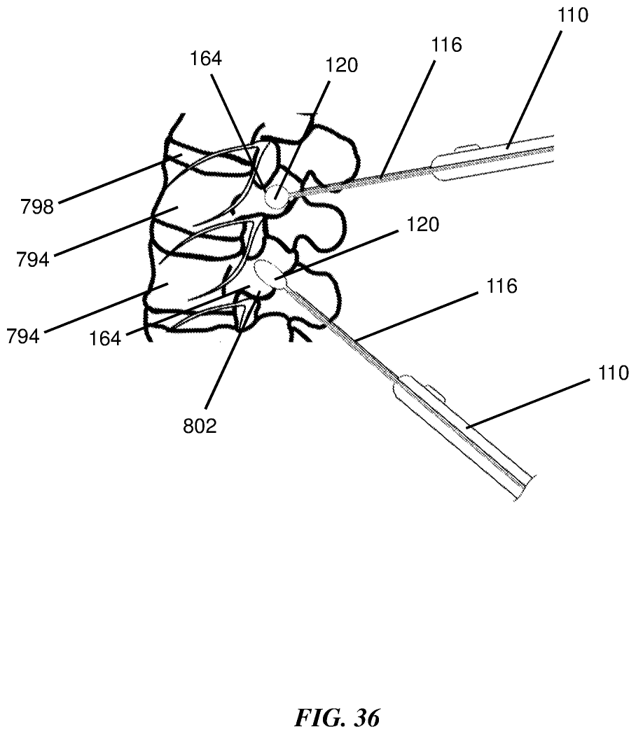

FIG. 36 illustrates how a probe 110 with an expandable balloon or basket tip 120 may be used to treat nerves at the lumbar facet joint 802. The cannula 116 may be inserted into a body via an upward posterior approach towards the nerves adjacent the facet joint 802 and a probe tip 120 with an elongate balloon or basket may be deployed and expanded to treat these nerves. Such an approach is shown with the lower probe 110 in FIG. 36 and the probe tip is oriented with its axis generally parallel to the surface of the facet bone. The elongate balloon or basket provides an active treatment region 750 along on the side of the balloon or basket and provides an elongated treatment region to ensure that the target nerves are treated. The electrode 736 may be configured such that the distal tip of the balloon or basket is not part of the active treatment region 750 to minimize the risk of treating material beyond the target tissue since the probe approaches the tissue generally parallel to the surface of the bone. The side of the balloon or basket with the active region 750 is pressed into facet joint with the side of the balloon or basket pressed onto the surface of the facet to conform to the facet surface and treat a broader area of the facet surface. The active treatment region 750 on the balloon or basket is placed in the region of a facet nerve such as a medial branch nerve or the dorsal ramus and on the surface of the facet. A balloon or basket with an active treatment region 750 on one side of the balloon or basket and not on the other side of the balloon or basket separates adjacent tissue from the active treatment region and focuses treatment on the target tissue while minimizing collateral effects on adjacent tissue.

Alternatively, a perpendicular approach may be made. In a perpendicular approach, the probe 110 is oriented somewhat downwardly and the axis of the probe 110 is oriented generally perpendicular to the surface of the facet at the location of the facet nerves (e.g. a medial branch nerve or the dorsal ramus nerve). This is shown by the upper probe 110 in FIG. 36. Such an approach would typically be made with a probe tip 120 which has a rounded end when expanded and which includes an electrode 736 and active treatment region 750 on the distal end of the probe tip 120. In this approach, the distal end of the probe tip 120 is pressed against the surface of the facet bone in the area of the facet nerves. This perpendicular approach is safer is it is less likely to heat and injure the dorsal root nerve or spinal nerve and because it provides an approach which avoids the mammillary process and ligament. Accordingly, the expandable probe tip 120 with an active treatment portion 750 on its distal end allows for treatment of facet nerves with less risk of injuring other surrounding structures. In both approaches, the expandable probe tip 120 is advantageous as it conforms to the surface of the facet bone and provides effective treatment over a desired area of the bone.

The balloon or basket probe tips 120 are thus advantageous as they provide a broader active treatment area along a particular portion of a larger probe tip 120. This allows a medical practitioner to provide treatment uniformly across a larger area or volume or target tissue. This increases the successful outcomes in treating such a broader portion of target tissue. Nerves which are more difficult to precisely target are more consistently treated. Tumors or other tissues are more evenly treated. Localized areas without sufficient treatment are avoided.

The above description of illustrated examples of the present invention, including what is described in the Abstract, is not intended to be exhaustive or to be limiting to the precise forms disclosed. While specific examples of the invention are described herein for illustrative purposes, various equivalent modifications are possible without departing from the broader scope of the present claims. Indeed, it is appreciated that specific example dimensions, materials, voltages, currents, frequencies, power range values, times, etc., are provided for explanation purposes and that other values may also be employed in other examples in accordance with the teachings of the present invention.

What is claimed is:

1. A high frequency electricity medical treatment probe comprising:
a probe body;
a cannula extending from a distal end of the probe body, the cannula having a lumen;
a probe tip disposed within the cannula lumen;
wherein the probe tip is movable proximally and distally within the cannula lumen such that the probe tip may be deployed from the cannula to a position exterior to a distal end of the cannula and such that the probe tip may be retracted into the cannula to a position interior to the cannula;
wherein the probe tip is expandable after deployment from the cannula to a diameter which is larger than a diameter than the cannula; and
an active treatment region on the expandable probe tip comprising an electrode which receives high frequency electrical energy from a power source; and
wherein, during use of the probe, the probe tip is inserted into a body tissue and transmits the high frequency electrical energy into a target tissue to treat the target tissue.

2. The probe of claim 1, wherein the probe tip has a first, collapsed state wherein the probe tip comprises a first diameter which fits within the cannula lumen and a second, expanded state wherein the probe tip has a second diameter which is larger than the first diameter and which is larger than an outside diameter of the cannula.

3. The probe of claim 1, wherein the probe tip comprises an ellipsoid shape having an axis oriented along a bore axis of the cannula lumen.

4. The probe of claim 3, wherein the active treatment region is located on a first side of the probe tip and wherein a second side of the probe tip opposite the first side of the probe tip does not comprise an active treatment region.

5. The probe of claim 4, wherein the active treatment region extends through an area which comprises approximately one third of the circumference of a middle of the probe tip.

6. The probe of claim 3, wherein the active treatment region is located on a distal end of the probe tip and wherein the portion of the probe tip proximal of the active treatment region does not comprise an active treatment region.

7. The probe of claim 6, wherein the active treatment region occupies the distal end of the probe tip and comprises between about one fourth and about one half of a length of the probe tip.

8. The probe of claim 1, wherein the probe tip comprises an expandable mesh of electrode wire which occupies a three dimensional volume to create an active treatment region.

9. The probe of claim 8, wherein the expandable mesh comprises a plurality of electrode wire loops which extend from a proximal point towards a distal end of the probe tip, bend around a distal end of the probe tip, and extend towards the proximal point, and wherein the plurality of electrode wires are oriented on different planes which generally intersect a longitudinal axis of the probe tip.

10. The probe of claim 9, wherein the probe tip further comprises an expandable balloon disposed inside of a volume defined by the electrode wire loops.

11. The probe of claim 8, wherein the probe tip expandable mesh comprises a first electrode wire which spirals in a helix about a probe tip longitudinal axis from a proximal point towards a distal end of the probe tip, bends around a distal end of the probe tip, and spirals in a helix about the probe tip longitudinal axis towards the proximal point.

12. The probe of claim 11, wherein the probe tip expandable mesh comprises a second electrode wire which spirals in a helix about a probe tip longitudinal axis from a proximal point towards a distal end of the probe tip, bends around a distal end of the probe tip, and spirals in a helix about the probe tip longitudinal axis towards the proximal point, wherein the first electrode wire comprises a right handed helix, and wherein the second electrode wire comprises a left handed helix.

13. The probe of claim 8, wherein the electrode wire is connected to an electrical lead wire at the probe tip, and wherein the electrical lead wire is shielded such that the electrical lead wire does not form an active treatment region.

14. The probe of claim 1, further comprising a retractor which is connected to the probe tip and configured to move the probe tip between a first position wherein the probe tip is disposed inside of the cannula lumen and a second position wherein the probe tip is deployed outside of the cannula lumen.

15. The probe of claim 1, further comprising a fluid pump connected to the probe tip via a fluid channel, and wherein the fluid pump is operable to pump fluid into the probe tip to expand the probe tip and is operable to receive fluid from the probe tip to collapse the probe tip.

16. A method for treating a target body tissue using a high frequency electricity medical treatment probe comprising:
selecting a high frequency electricity medical treatment probe comprising:
a probe body;
a cannula extending from a distal end of the probe body, the cannula having a lumen;
an expandable probe tip disposed within the cannula lumen;
wherein the probe tip is movable proximally and distally within the cannula lumen such that the probe tip may be deployed from the cannula to a position exterior to a distal end of the cannula and such that the probe tip may be retracted into the cannula to a position interior to the cannula;
wherein the probe tip is expandable after deployment from the cannula to a diameter which is larger than a diameter than the cannula; and
wherein the probe tip comprises an active treatment region on the expandable probe tip comprising an electrode which receives radio frequency energy and transmits the radio frequency energy into a target tissue;
inserting the cannula into body tissue to position the distal end of the cannula adjacent a target tissue;
moving the probe tip to a position exterior to the lumen of the cannula such that the probe tip extends distally from the distal end of the cannula;
expanding the probe tip such that an expanded diameter of the probe tip is greater than a diameter than the cannula; and
delivering high frequency electricity to the probe tip electrode to thereby treat the target tissue.

17. The method of claim 16, wherein the active treatment region is located on a first, lateral side of the probe tip, wherein a second side of the probe tip opposite the first side of the probe tip does not comprise an active treatment region, and wherein the method comprises placing the side of the probe tip adjacent a target tissue such that the electrode treats target tissue adjacent the active treatment region and such that the opposite side of the probe tip isolates adjacent tissue from the active treatment region to shield the adjacent tissue from treatment.

18. The method of claim 16, wherein the active treatment region is located on a distal end of the probe tip, wherein the portion of the probe tip proximal of the active treatment region does not comprise an active treatment region, and wherein the method comprises placing the distal end of the probe tip adjacent a target tissue such that the electrode treats target tissue adjacent the active treatment region and such that a proximal end of the probe tip isolates adjacent tissue from the active treatment region to shield the adjacent tissue from treatment.

19. The method of claim 16, wherein the method comprises inserting the cannula into a bone, moving the probe tip from the cannula into a core of the bone, expanding the probe tip into the core of the bone, and delivering high frequency electricity to the probe tip electrode to thereby treat target tissue within the core of the bone.
